(19) United States
(12) Patent Application Publication
(10) Pub. No.: US 2026/0354839 A1
(43) Pub. Date: Nov. 12, 2026

(54) MINIMALLY INVASIVE TRANS-FACET SURGICAL INSTRUMENTS

(71) Applicant: Amr Osman El-Naggar, Somerset, KY (US)

(72) Inventor: Amr Osman El-Naggar, Somerset, KY (US)

(21) Appl. No.: 19/668,230

(22) Filed: May 5, 2026

Related U.S. Application Data
(60) Provisional application No. 63/800,669, filed on May 6, 2025.

(51) Int. Cl. A61B 17/16; A61B 17/00; A61B 17/17; A61B 17/32; A61B 17/56

(52) U.S. Cl. CPC A61B 17/1615; A61B 17/00234; A61B 17/1631; A61B 17/1671; A61B 17/1757; A61B 17/32; A61B 17/56; A61B 2017/00238; A61B 2017/320008; A61B 2017/564

(57) ABSTRACT

Systems and methods for spinal surgery are disclosed. The systems and methods are directed to instruments for forming an access corridor through a facet joint. The systems may include a guidance rod, a tubular dilator, an anchoring dilator, an introducer sleeve, and a disc preparation drill. Forming the access corridor may include inserting a tapered reamer into the anchoring dilator.

### CROSS-REFERENCE TO RELATED APPLICATIONS

This application claims the benefit of U.S. Provisional Application No. 63/800,669, filed May 6, 2025, the contents of which are incorporated herein by reference in their entirety.

### TECHNICAL FIELD

The present disclosure relates generally to instruments for use in spinal surgery and more particularly to techniques for performing minimally invasive trans-facet spinal surgery.

### BACKGROUND

Spinal surgery can involve addressing conditions including stenosis, herniated discs, fractures, spinal deformities, spondylolisthesis, or degenerative disc diseases. Access to the effected spinal anatomy can involve removing all or part of the inferior articular process and/or superior articular process. These bony articular processes form the zygapophyseal facet joints, which constitute key components of the posterior spinal column and provide stability while allowing movement between vertebrae. In the context of lumbar fusion surgery, for example, conventional approaches include posterior lumbar interbody fusion (PLIF), trans-foraminal lumbar interbody fusion (TLIF), anterior lumbar interbody fusion (ALIF), direct lateral interbody fusion (DLIF), and extreme lateral interbody fusion (XLIF), among others. Although these approaches may be suitable in certain instances, they may present certain drawbacks, including exposure of the neural elements, risk of nerve root injury, risk of epidural scarring or arachnoiditis, risk of infection, and significant disturbance of the paraspinal muscles. A need therefore exists for improved instruments and techniques for performing spinal surgeries that may overcome one or more of these drawbacks associated with existing approaches.

### SUMMARY

The present disclosure provides instruments and techniques for performing trans-facet spinal surgery. In some examples, the disclosed instruments and techniques facilitate performance of trans-facet spinal surgery that preserves aspects of both the inferior and superior articular processes of a vertebra.

In some examples, the systems and methods described herein relate to a surgical reamer system including a reamer for forming a surgical corridor in a patient. The reamer includes a longitudinal body extending between a proximal portion and a distal portion and define a central lumen extending longitudinally therethrough. The distal portion includes a tapered region that extends between a base and a distal tip and tapers toward the distal tip. At least a portion of the tapered region includes a cutting edge for removing bone along the surgical corridor. The distal tip defines an opening in fluid communication with the central lumen.

In some examples, the cutting edge of the reamer extends helically about the tapered region. In some examples, the cutting edge of the reamer extends along an entire length of the tapered region. In some examples, the cutting edge of the reamer is axially spaced from the distal tip. In some examples, the cutting edge of the reamer is steel-coated.

In some examples, the distal portion of the reamer defines a groove extending longitudinally along an outer surface of the distal portion. In some examples, the groove extends through at least a portion of the cutting edge. In some examples, the groove extends to the base of the tapered region.

In some examples, the base of the tapered region has a diameter that is 3 mm larger than a diameter of the distal tip. In some examples, a distance between the base of tapered region and the distal tip of the distal portion has a length of 6 cm.

In some examples, the longitudinal body of the reamer includes a barreled portion having a diameter greater than a diameter of the base of the tapered region.

In some examples, the reamer further includes a handle for rotating the reamer about a longitudinal axis.

In some examples, the surgical reamer system further includes a joint stripper for removing soft tissue from a patient's joint. The joint stripper includes a longitudinal body extending between a proximal portion and a distal portion and defines a central lumen extending longitudinally therethrough. The distal portion of the joint stripper includes a plurality of distally extending protrusions. Each protrusion includes a cutting edge for removing soft tissue from the patient's joint.

In some examples, the joint stripper further includes a handle for rotating the joint stripper about a longitudinal axis.

In some examples, the joint stripper is disposable within an anchoring dilator. In such examples, the anchoring dilator includes a proximal portion and a distal portion and defines a central lumen extending longitudinally therethrough, where the distal portion includes a plurality of distally extending protrusions for frictionally engaging patient tissue.

In some examples, the systems and methods described herein relate to a surgical dilator system for enlarging a corridor in a patient. The surgical dilator system includes: a guidance rod extending between a proximal end and a tapered distal tip; a first tubular dilator extending between a proximal portion and a distal portion and defining a central lumen extending longitudinally therethrough, wherein the central lumen is sized and configured to receive the guidance rod; and an anchoring dilator extending between a proximal portion and a distal portion and defining a central lumen extending longitudinally therethrough, wherein the distal portion includes a plurality of distally extending protrusions for frictionally engaging patient tissue, wherein the central lumen of the anchoring dilator is sized and configured to receive the guidance rod and first tubular dilator.

In some examples, the surgical system further includes one or more additional tubular dilators disposed radially outward of the first tubular dilator.

In some examples, the plurality of distally extending protrusions include a plurality of serrated edges. In some examples, the plurality of distally extending protrusions extend circumferentially about the distal portion of the anchoring dilator. In some examples, the plurality of distally extending protrusions extend at an oblique angle relative to a longitudinal axis of the anchoring dilator. In some examples, the plurality of distally extending protrusions have lengths of 4 mm.

In some examples, the proximal portion of the anchoring dilator includes a handle.

In some examples, the surgical dilator system further includes an introducer extending between a proximal portion and a distal portion and defining a central lumen extending longitudinally therethrough, where the central lumen of the introducer is sized and configured to receive the anchoring dilator.

In some examples, the proximal portion of the introducer includes a handle.

In some examples, the surgical dilator system further includes a universal dilator. The universal dilator extends between a proximal portion and a distal portion and defining a central lumen extending longitudinally therethrough. The distal portion of the universal dilator includes a tapered region that tapers toward a distal tip of the distal portion. The distal tip defines an opening in fluid communication with the central lumen.

In some examples, the tapered region of the universal dilator defines a conical shape.

In some examples, central lumen of the universal dilator is sized and configured to receive the guidance rod.

In some examples, the universal dilator further includes a handle disposed at the proximal portion of the universal dilator.

In some examples, the systems and methods described herein relate to a kit for forming a surgical corridor in a patient. The kit includes: a first tubular dilator extending between a proximal portion and a distal portion and defining a central lumen extending longitudinally therethrough, wherein the central lumen is sized and configured to receive a guidance rod; an anchoring dilator extending between a proximal portion and a distal portion and defining a central lumen extending longitudinally therethrough, wherein the distal portion includes a plurality of distally extending protrusions for frictionally engaging patient tissue, wherein the anchoring dilator is disposed radially outward of the first tubular dilator; and a reamer including a longitudinal body extending between a proximal portion and a distal portion and defining a central lumen extending longitudinally therethrough, wherein the distal portion includes a tapered region that extends between a base and a distal tip and tapers toward the distal tip, wherein at least a portion of the tapered region includes a cutting edge for removing bone along the surgical corridor, and wherein the distal tip defines an opening in fluid communication with the central lumen, the central lumen configured to accommodate the guidance rod.

In some examples, the kit further includes an introducer extending between a proximal portion and a distal portion and defining a central lumen extending longitudinally therethrough. The introducer is disposed radially outward of the anchoring dilator.

In some examples, the systems and methods described herein relate to a method of forming a surgical corridor in a patient. The method includes: inserting a guidance rod through an incision site and advancing the guidance rod toward a treatment location of a patient such that a distal tip of the guidance rod contacts tissue superficial relative to the treatment location, wherein the path of the guidance rod defines a surgical corridor; inserting a reamer over the guidance rod to form the surgical corridor, the reamer including a tapered region that tapers from a base of the tapered region toward a distal tip of the tapered region, the tapered region including a cutting edge; removing the reamer from the surgical corridor; advancing a first tubular dilator over the guidance rod, thereby expanding the surgical corridor and incision site; advancing an anchoring dilator over the first tubular dilator; and frictionally engaging the patient's tissue with a plurality of distally extending protrusions defined on a distal portion of the anchoring dilator.

In some examples, the method further includes rotating the reamer such that the cutting edge removes tissue along the surgical corridor.

In some examples, the method further includes advancing one or more additional tubular dilators over the first tubular dilator, thereby further expanding the surgical corridor and incision site.

In some examples, the method further includes forming the surgical corridor includes forming a surgical corridor through a facet joint of a patient.

In some examples, the method further includes removing disc material from a disc space of the patient via one or more instruments advanced through the surgical corridor.

In some examples, the surgical corridor intersects a middle aspect of the facet joint between an inferior articular process of a vertebra above the disc space and a superior articular process of a vertebra below the disc space. In some examples, aspects of the inferior and superior articular processes are preserved on both sides of the surgical corridor so as to define lateral sides of the surgical corridor.

In some examples, the method further includes inserting an intervertebral disc into the disc space. In some examples, the method further includes inserting a graft material into the disc space. In some examples, the method further includes inserting a graft material into the surgical corridor. In some examples, the graft material is deposited into the surgical corridor from the disc space to the incision site.

### BRIEF DESCRIPTION OF THE DRAWINGS

FIGS. 1A-1C are side views of various exemplary access cannulas. FIG. 1A is a side view of an exemplary access cannula. FIG. 1B is a detail view of the exemplary access cannula shown in FIG. 1A. FIG. 1C is a detail view of another exemplary access cannula.

FIGS. 2A-2B are side views of an exemplary guide rod.

FIG. 3 is a side view of the exemplary guide rod shown in FIG. 2A in use with the exemplary access cannula shown in FIG. 1A.

FIG. 4 is a side view of an exemplary universal dilator.

FIGS. 5A-5E are various views of an exemplary reamer. FIG. 5A is a side view of the reamer. FIG. 5B is a side view of the reamer according to a specific example. FIG. 5C is a detailed view of the reamer shown in FIG. 5A. FIG. 5D is a detailed view of the reamer shown in FIG. 5B. FIG. 5E is a further detailed view of the reamer shown in FIG. 5C.

FIGS. 6A-6B are various views of another exemplary reamer. FIG. 6A is a side view of the tapered reamer. FIG. 6B is a detailed view of the reamer shown in FIG. 6A.

FIG. 7 is a side view of another exemplary reamer.

FIG. 8 is a side view of another exemplary reamer.

FIG. 9 is a detailed side view of another exemplary reamer.

FIG. 10 is a side view of an exemplary surgical dilator system, including an anchoring dilator.

FIGS. 11A-11C are side views of various exemplary dilators.

FIGS. 12A-12C are side views of various anchoring dilators. FIG. 12A is a side view of the anchoring dilator shown in FIG. 10. FIG. 12B is a detail view of the anchoring dilator of FIG. 12A. FIG. 12C is a detail view of another exemplary anchoring dilator. FIG. 12D is a bottom view of the anchoring dilator shown in FIG. 12A.

FIG. 13 is a side view of another exemplary surgical dilator system, including an anchoring dilator.

FIGS. 14A-14B are side views of the anchoring dilator shown in FIG. 13.

FIG. 15 is a side view of an exemplary surgical reamer system, including a reamer and a joint stripper.

FIGS. 16A-16C are side views of various joint strippers. FIG. 16A is a side view of the joint stripper shown in FIG. 15. FIG. 16B is a detail view of the joint stripper of FIG. 16A. FIG. 16C is a detail view of another exemplary joint stripper.

FIG. 17 is a side view of an exemplary anchoring dilator shown in FIG. 12A in use with an exemplary joint stripper shown in FIG. 16A.

FIG. 18 is a side view of an exemplary disc preparation drill.

FIGS. 19A-19C are detail side views of various example preparations drills.

FIGS. 20A-20C are side views of an exemplary articulating stylet. FIG. 20A is a side view of the exemplary articulating stylet extending axially relative to a longitudinal axis. FIG. 20B is a side view of the exemplary articulating stylet articulating relative to the longitudinal axis. FIG. 20C is a side view of the exemplary articulating stylet further articulating relative to the longitudinal axis.

FIGS. 21A-21B are side views of the exemplary disc preparation drill in use with an exemplary anchoring dilator. FIG. 21A is a side view of the exemplary disc preparation drill extending axially through the exemplary anchoring dilator toward the treatment site. FIG. 21B is a side view of the exemplary disc preparation drill articulating within the treatment site and cutting tissue therein.

FIGS. 22A-22D are side views of various exemplary access cannulas. FIG. 22A is a side view of an exemplary access cannula. FIG. 22B is a side view of another exemplary access cannula with the handle removed. FIG. 22C is a detail view of the exemplary access cannula shown in FIG. 22A. FIG. 22D is a detail view of another exemplary access cannula. FIG. 22E is a detail view of the distal portion of the exemplary access cannula of FIG. 22A or FIG. 22B with the stylet removed.

In the drawings, like reference numbers and designations in the various drawings indicate like elements.

The detailed description is set forth with reference to the accompanying drawings. The drawings are provided for purposes of illustration only and merely depict example implementations of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components.

Various implementations may utilize elements or components other than those illustrated in the drawings, and some elements and/or components may not be present in various implementations. The use of singular terminology to describe a component or element may, depending on the context, encompass a plural number of such components or elements and vice versa.

### DETAILED DESCRIPTION

In the following description, specific details are set forth describing some implementations consistent with the present disclosure. Numerous specific details are set forth in order to provide a thorough understanding of the implementations. It will be apparent, however, to one skilled in the art that some implementations may be practiced without some or all of these specific details. The specific implementations disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one implementation may be incorporated into other implementations unless specifically described otherwise or if the one or more features would make an implementation non-functional. In some instances, well known methods, procedures, and components have not been described in detail so as not to unnecessarily obscure aspects of the implementations.

The following description of certain examples of the inventive concepts should not be used to limit the scope of the claims. Other examples, features, aspects, implementations, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing examples. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but that conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The terms “proximal” and “distal” as used herein refer to regions of a reamer, dilator, joint stripper, guidance rod, or other surgical instrument or device. “Proximal” means that region closest to the handle of the device or the operator of the device, while “distal” means that region farthest away from the handle of the device or the operator of the device.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Systems and methods for forming a surgical access corridor are provided. In some examples, the access corridor extends between an incision entry point on the surface of the patient's skin and extends toward a surgical target site (also referred to herein as a “treatment location”). For example, the surgical target site can encompass the disc space between two adjacent vertebral discs in a patient's spine. In some specific examples, as further described herein, the access corridor can be formed so as to facilitate surgical access to the disc space of a patient while maintaining bony margins about the zygapophyseal facet joint. Exemplary methods of performing minimally invasive trans-facet spinal surgery, also referred to herein the “ZLIF” (or “Z-LIF”) operation, are disclosed in U.S. Pat. No. 11,925,566, entitled “Minimally Invasive Robotic Assisted Trans-Facet Lumbar Interbody Fusion,” U.S. patent application Ser. No. 18/428,295, entitled “Minimally Invasive Robotic Assisted Trans-Facet Lumbar Interbody Fusion,” and U.S. patent application Ser. No. 18/953,873, entitled “Minimally Invasive Trans-Facet Spinal Surgery,” the disclosures of which are incorporated herein by reference in their entirety.

The systems and methods for forming the access corridor can include a series of instruments for aligning the systems, preparing the patient's tissue for surgery, removing tissue from the patient to form the access corridor (for example, through the zygapophyseal facet joint), and preparing the target site for further surgery. Accordingly, the systems can include a guidance rod, one or more dilators, one or more tapered reamers, introducer sleeves, and handles, as well as other conventional instruments. The various instruments can be employed at one or more stages of the surgical procedure, as appropriate. Furthermore, various surgical instruments provided herein can be manually operated. Accordingly, certain medical procedures such as the ZLIF can be performed under fluoroscopic guidance, computed tomography (“CT”), O-Arm, or spine navigation guidance. However, it is also contemplated herein that a robotic surgical guidance system can optionally be used to handle and manipulate any one or more of the described surgical instruments.

### Example Systems and Instruments

### Access Cannula and Guidance Rod

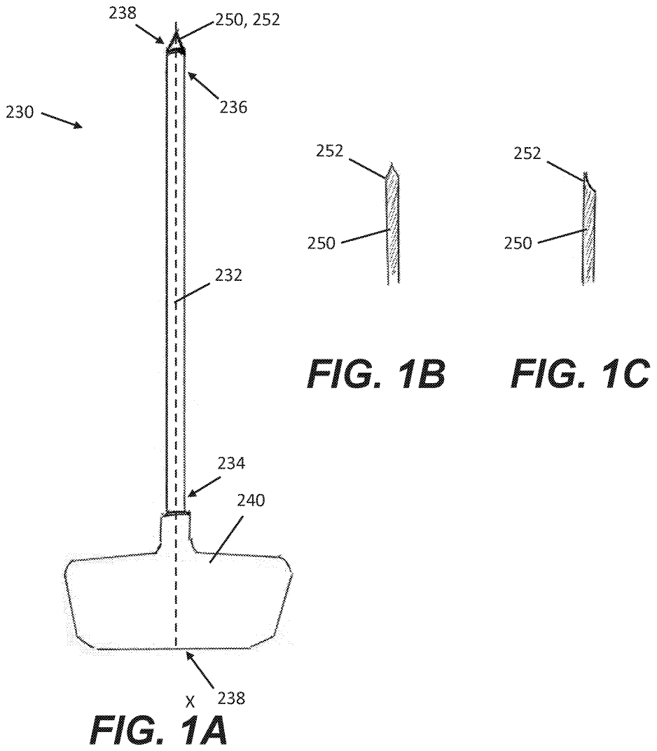

In one aspect, the systems and methods can include one or more surgical instruments for forming a surgical corridor in a patient. In various examples, a primary access cannula 230 can be used to facilitate placement of a surgical guidance rod 140. Referring to FIGS. 1A-1C, an example access cannula 230 is provided. As shown, the access cannula 230 includes a longitudinal body 232 that extends between a proximal portion 234 of the access cannula 230 and a distal portion 236 of the access cannula 230. A central lumen 238 extends longitudinally through an entire length of the longitudinal body 232 of the access cannula 230 so as to allow translation of a guidance rod 140 therethrough. Thus, the inner diameter of the central lumen 238 can be sized and configured to accommodate translational movement of the guidance rod 140 therethrough. Accordingly, the central lumen 238 can range from about 1 mm to about 5 mm, depending upon the diameter of the guidance rod 140 to be slidably disposed therein. In the illustrated example, the central lumen 238 of the access cannula 230 has a diameter of about 2 mm.

As shown in FIG. 1A, a stylet 250 having a pointed distal tip 252 can be inserted into the central lumen 238 of the access cannula 230. While the example access cannula 230 illustrated in FIG. 1A includes a stylet 250 having a diamond-shaped pointed distal tip 252 (shown in greater detail in FIG. 1B), other shaped distal tips 252 are contemplated. For example, FIG. 1C provides an example stylet 250 with a pointed distal tip 252 having a beveled shape.

Advantageously, the pointed distal tip 252 allows a user to pierce the patient's tissue at an incision entry point and gradually advance the access cannula 230 intracorporeally toward the target surgical site. Importantly, the trajectory of the access cannula 230 will approximately define a surgical path to the target surgical site coincident with a longitudinal axis X of the access cannula 230. Accordingly, a preoperative planning scheme that can include preoperative magnetic resonance imaging (“MRI”) or a myelogram performed along with a preoperative computed tomography (“CT”) scan may be implemented. Data gathered from these imaging modalities can be used to inform placement of the incision entry point, the trajectory of the access cannula 230, and thus the desired surgical path to the target surgical site. In some cases, the access cannula 230 is advanced toward the target surgical site (e.g., to be docked on the posterior aspect of the facet joint) under anteroposterior (“AP”) imaging and/or lateral fluoroscopy.

The example access cannula 230 shown in FIG. 1A includes a handle 240 that can be used to advance the access cannula 230 through the patient's tissue and toward the surgical target site. As shown, the central lumen 238 extends through the entire length of the handle 240. It is noted that the access cannula 230 may instead be driven by a robotic surgical guidance system. Indeed, although various surgical instruments provided herein can optionally include handles, each surgical instrument can instead be manipulated via a robotic surgical guidance system.

The illustrated access cannula 230 has a length of about 16 cm, as measured from the handle 240 to the pointed distal tip 252 of the stylet 250. In further examples, the length of the access cannula 230 can range from about 10 cm to about 20 cm.

As illustrated in FIG. 1A, in some examples the access cannula 230 has a constant inner and/or outer diameter along the entire length of the longitudinal body 232 of the access cannula 230. In some examples, the access cannula 230 has a decreasing inner and/or outer diameter between the proximal and distal ends of the longitudinal body 232. In some examples, the outer diameter of the access cannula 230 ranges from about 1 mm to about 10 mm, In further examples, the outer diameter of the access cannula 230 ranges from about 2 mm to about 6 mm. In some examples, the outer diameter of the access cannula 230 is about 2 mm, about 3 mm, about 4 mm, about 5 mm, or about 6 mm. However, as provided herein, the dimensions of the access cannula 230 can vary depending on such factors as the size of the patient, the distance between the incision entry point and the surgical target site, and the selection of surgical instruments and devices.

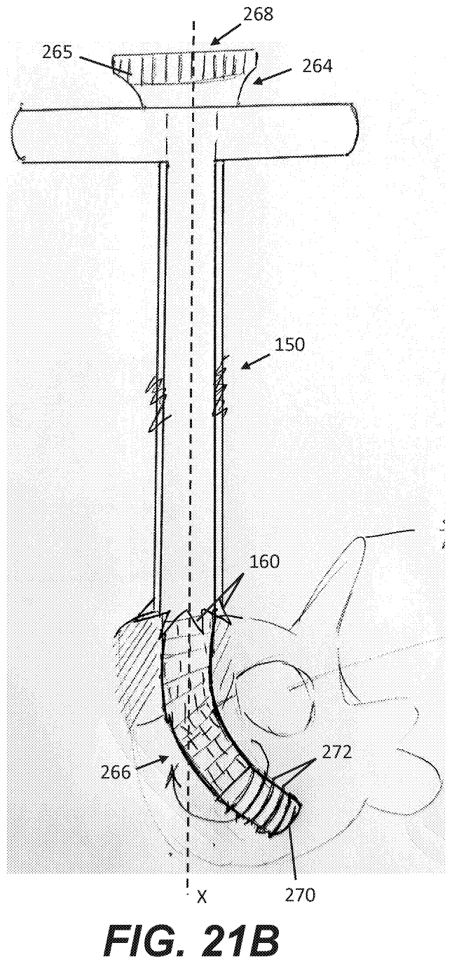

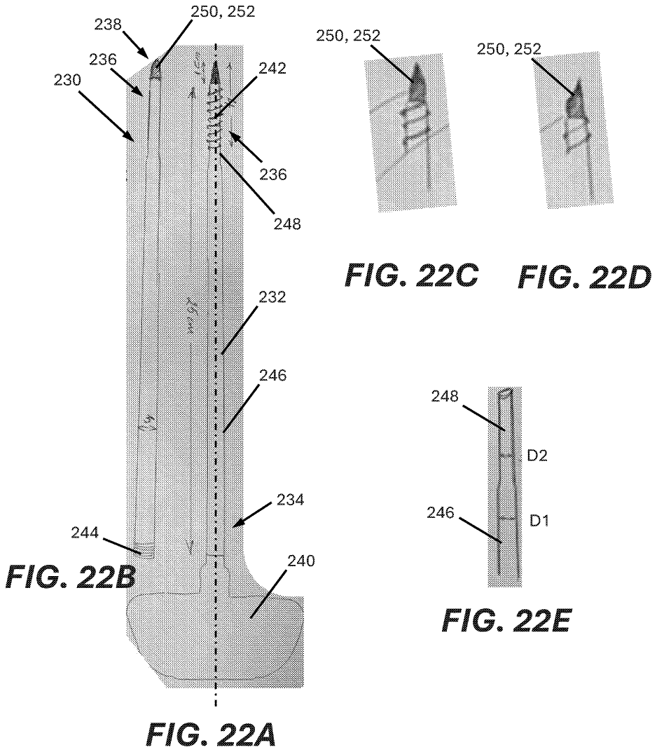

Referring to FIGS. 22A-22E, another example access canula 230 is provided. The access cannula 230 of FIGS. 22A-22E includes similar features to the access cannula 230 of FIGS. 1A-1C, accordingly, like reference numbers are used to identify like elements. The differences between the access cannula 230 of FIGS. 1A-1C and the access cannula 230 of FIGS. 22A-22E are provided herein.

As shown in FIG. 22A, the access cannula 230 includes include a threaded portion 242 extending along the distal portion 236 of the longitudinal body 232. The threaded portion 242 is sized and configured to releasably couple the access cannula 230 with the lumbar body (e.g., facet joint and/or disc space). For example, the threaded portion 242 can be used to manually engage and drive the access cannula 230 into the mid-facet region of the superior and inferior process and/or into the disc space. Fixing the access cannula 230 to the facet joint and/or disc space helps to ensure that the position of the access cannula 230 is fixed relative to the facet joint/disc space. As described herein, in some examples the access cannula 230 can remain in place during the procedure. Fixing the access cannula 230 helps to ensure that the access cannula 230 does not move or shift during the procedure. For example, fixing the access cannula 230 to the facet joint using the threaded portion 242 helps to ensure that the access cannula 230 does not move or shift during the drilling of the midfacet region to create the access corridor. In some examples, without coupling the access cannula 230 to the facet joint, vibration created by the drill bit may inadvertently displace the access cannula 230.

In some examples, the threaded portion 242 includes one or more helical protrusions extending about and along the longitudinal body 232. The protrusions of the threaded portion 242 may have any suitable profile, pitch, lead, and direction. In some examples, the threaded portion 242 comprises a self-forming or self-tapping thread configured to form, cut, or displace material of the facet joint and/or disc material during insertion. The self-tapping threaded portion 242 may include one or more cutting edges, flutes, or sharpened thread crests.

Similar to the access cannula 230 of FIGS. 1A-1C, the access cannula 230 includes a longitudinal body 232 that extends between the proximal portion 234 and distal portion 236 of the access cannula 230, with a central lumen 238 extending therethrough. In some examples, the threaded portion 242 extends from the distal end of the access cannula 230 and/or the distal opening of the central lumen 238, and toward the proximal portion 234. In some examples, the threaded portion 242 is offset from the distal end/opening of the access cannula 230. As illustrated in FIG. 22A, in the threaded portion 242 extends a length (L) along the longitudinal body 232 from the distal end of the access cannula 230 and/or the distal opening of the central lumen 238 toward the proximal portion 234, In some examples, the length (L) ranges from about 5 mm to about 5 cm. In some examples, the length (L) ranges from about 1 cm to about 4.5 cm. In some examples, the length (L) ranges from about 2 cm to about 4 cm. In some examples, the length (L) ranges from about 3 cm to about 4 cm. In some examples, the length (L) is about 4 cm.

As illustrated in FIG. 22A, the access cannula 230 includes a handle 240 at its proximal end. As provided in FIG. 22B, showing an example access cannula 230 without the threaded portion 242, the handle 240 can be removably coupled to the longitudinal body 232 of the access cannula 230. For example, as illustrated in FIG. 22B, the handle 240 can be coupled to the longitudinal body 232 of the access cannula 230 as a proximal threaded portion 244. The proximal threaded portion 244 can extend from the proximal end of the access cannula 230 and/or the proximal opening of the central lumen 238, and toward the distal portion 236. The handle 240 can be removably coupled to the longitudinal body 232 by any other connector, including, for example, a bayonet (twist-lock) connector, snap-fit connection, interference or press-fit connection, collet-based connector, compression fitting, detent-based connection, magnetic coupling, or a keyed coupling configured to align and secure the handle 240 relative to the longitudinal body 232 of the access cannula 230. In some examples, the connector may be configured to permit axial attachment and removal of the handle 240 from the longitudinal body 232 while maintaining a secure engagement of the access cannula 230 with the facet joint.

Similar to the access cannula 230 illustrated in FIGS. 1A-1C, as shown in FIGS. 22A and 22B, and the enlarged view of the distal portion 236 of the access cannula 230 shown in FIGS. 22C and 22D, a stylet 250 having a pointed distal tip 252 can be inserted into the central lumen 238 of the access cannula 230. For example, the stylet 250 can have a diamond-shaped distal tip 252 (as illustrated in FIG. 22C), a beveled shape (as illustrated in FIG. 22D), or any other suitable shape that allows the user to pierce the patient's tissue.

In some examples, the cannula 230 illustrated in FIGS. 22A-22E has a length similar to the length of the access cannula 230 illustrated in FIGS. 1A-1C. In another example, because the access cannula 230 of FIGS. 22A-22E is configured to be advanced/received within the facet joint and/or disc space, the access cannula 230 is longer than the access cannula 230 illustrated in FIGS. 1A-1C. For example, in some implementations, the access cannula 230 has a length of about 26 cm, as measured from the handle 240 to the pointed distal tip 252 of the stylet 250, or about 25 cm as measured from the handle 240 to the distal end of the longitudinal body 232. In further examples, the length of the handle 240 can range from about 10 cm to about 30 cm.

As described herein, in some examples, the access cannula 230 has a decreasing inner and/or outer diameter along all or a portion of the entire length of the longitudinal body 232 of the access cannula 230. In some examples, as illustrated in FIGS. 22A, 22B, and 22D, the longitudinal body 232 can include a large diameter portion 246 and a small diameter portion 248, having a diameter less than the diameter of the large diameter portion 246. The small diameter portion 248 extends between the distal end of the longitudinal body 232 and the large diameter portion 246. The small diameter portion 248 can correspond with the length of the longitudinal body 232 sized and configured to be advanced within the facet joint and/or disc space. In some examples, as illustrated in FIG. 22A, the threaded portion 242 is provided along the small diameter portion 248. In some examples, the threaded portion 242 extends along only a portion of the length of the small diameter portion 248.

In some examples, the outer diameter (D1) of the large diameter portion 246 ranges from about 1 mm to about 10 mm and the outer diameter (D2) of the small diameter portion 248 ranges from 0.5 mm to about 9 mm. In some examples, the diameter (D1) of the large diameter portion 246 ranges from about 2 mm to about 6 mm, from about 2 mm to about 5 mm. In some examples, the diameter (D1) of the large diameter portion 246 is about 2 mm, about 3 mm, about 4 mm, about 5 mm, or about 6 mm. In some examples, the outer diameter (D2) of the small diameter portion 248 ranges from about 1 mm to about 4 mm, from about 2 mm to about 3 mm. In some examples the diameter (D2) of the small diameter portion 248 is about 1 mm, about 2 mm, about 3 mm, about 4 mm, or about 5 mm. In some examples, the diameter (D1) of the large diameter portion 246 is about 5 mm, and the diameter (D2) of the small diameter portion 248 is about 3 mm. In some examples, the diameter (D1) of the large diameter portion 246 is about 4 mm, and the diameter (D2) of the small diameter portion 248 is about 2 mm.

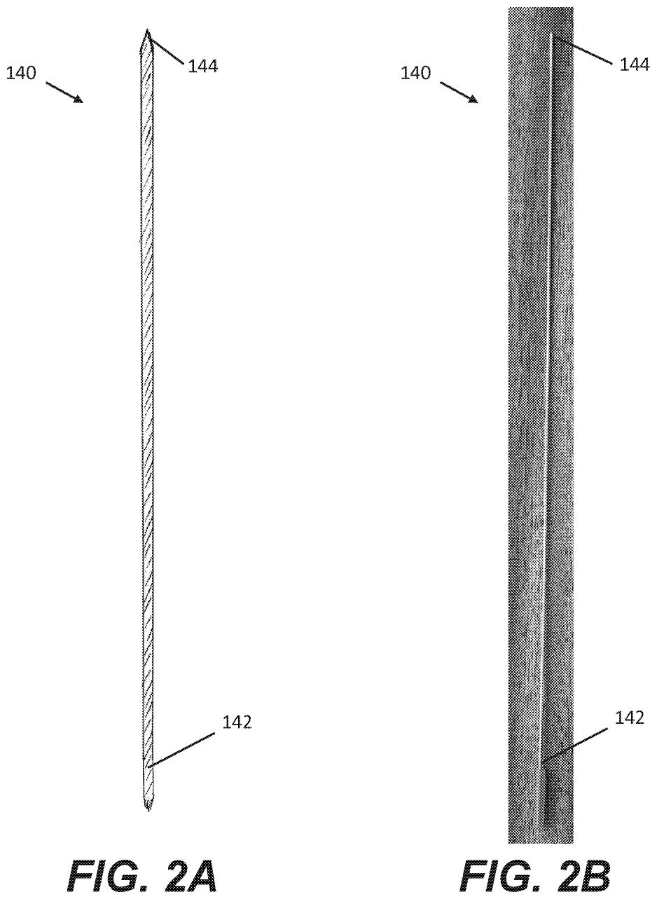

FIGS. 2A and 2B provide example guidance rods 140 for insertion into the access cannula 230. As shown in FIG. 2A, the guidance rod 140 extends between a proximal end 142 and a distal tip 144. The distal tip 144 is pointed to facilitate anchoring into the patient's tissue once the guidance rod 140 is advanced along the central lumen 238 and makes contact with patient tissue. In the example illustrated in FIG. 2A, the proximal end 142 of the guidance rod 140 is also pointed. However, FIG. 2B shows an example guidance rod 140 having a blunted proximal end 142.

Although the example guidance rods 140 shown in FIGS. 2A-2B have lengths of about 30 cm and diameters of about 1 mm, the dimensions of the guidance rod 140 can vary depending on such factors as the size of the patient, the distance between the incision entry point and the surgical target site, and the selection of surgical instruments and devices. For example, the length of the 140 can range from about 15 cm to about 50 cm. In some examples, the length of the 140 can range from about 20 cm to about 40 cm. Further, the diameter of the guidance rod 140 can range from about 0.5 mm to about 3 mm.

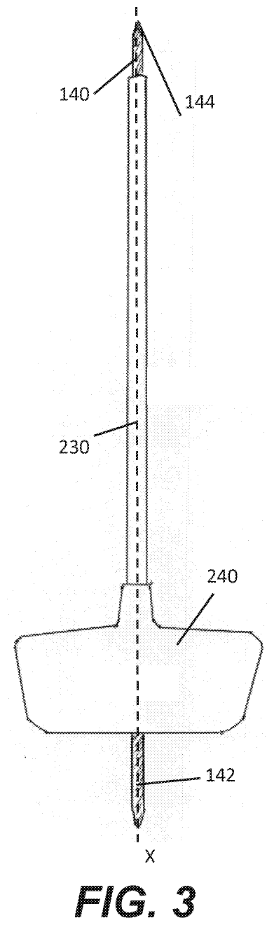

FIG. 3 shows an example access cannula 230 in use with an example guidance rod 140. Once the access cannula 230 has been placed, the stylet 250 is removed to make way for the guidance rod 140 to be advanced through the central lumen 238 along the longitudinal axis X. As further described herein, the guidance rod 140 serves to define the surgical path. Accordingly, various other instruments described herein can subsequently be advanced over the guidance rod 140 and translated along the length of the guidance rod 140 toward the target surgical site. Advantageously, proper positioning of the guidance rod 140 can thus reduce the likelihood that subsequent instruments advanced over the guidance rod 140 will encroach upon surrounding tissue structures. For example, when performing trans-facet surgeries, it can be advantageous to anchor the guidance rod 140 between an inferior articular process of a vertebra above the disc space and a superior articular process of a vertebra below the disc space such that bony material substantially extends around all sides of the guidance rod 140. For example, the guidance rod 140 can be anchored such that the subsequently formed surgical corridor intersects a middle aspect of the facet joint.

Thus, the surgical corridor subsequently formed along the surgical path defined by the guidance rod 140 is positioned to preserve aspects of the inferior and superior articular processes on either side of the surgical corridor so as to define lateral sides of the surgical corridor. Accordingly, sensitive neural elements are less likely to be disturbed during formation of the surgical corridor into the facet joint. Once the guidance rod 140 is anchored into the patient's tissue, the access cannula 230 can be withdrawn, leaving the guidance rod 140 in place.

### Universal Dilator

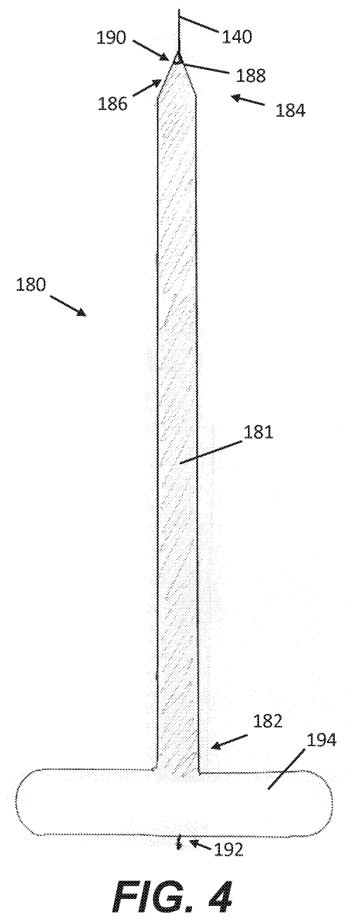

In some examples, one or more of the systems and methods provided herein can include a universal dilator 180 (alternatively referred to as a “one-step” dilator) for enlarging the surgical corridor following placement of the guidance rod 140. This allows the incision to be continued deeper through the fascia. An example universal dilator 180 is provided in FIG. 4. As shown in FIG. 4, the universal dilator 180 has a tubular body 181 that extends between a proximal portion 182 and a distal portion 184 and defines a central lumen 192 extending longitudinally at least partially therethrough. The distal portion 184 includes a tapered region 186 that tapers toward a distal tip 188 at the most distal extent of the distal portion 184. For example, the tapered region 186 can define a conical shape. As shown in FIG. 4, the distal portion 184 defines an opening 190 that is continuous with the central lumen 192.

As provided herein, the central lumen 192 can be sized and configured to accommodate translational movement along the guidance rod 140. Accordingly, the opening 124 can range from about 0.5 mm to about 5 mm depending upon the diameter of the guidance rod 140 to be slidably disposed therein. Specifically, the opening 124 of the example universal dilator 180 illustrated in FIG. 4 is about 2 mm.

Advantageously, the tapered shape of the tapered region 186 facilities gradual dilation of the patient tissue surrounding the universal dilator 180 as the universal dilator 180 is advanced axially along the guidance rod 140 toward the target surgical site. Use of the universal dilator 180 is particularly advantageous in the absence of a robotic surgery guidance system.

In some examples, including the illustrated example, the universal dilator 180 includes a handle 194 at the proximal portion 182 of the universal dilator 180. As shown, the central lumen 192 extends through the entire length of the universal dilator 180 such that a guidance rod 140 inserted into the central lumen 192 can extend proximally through the handle 194 and distally through the opening 190.

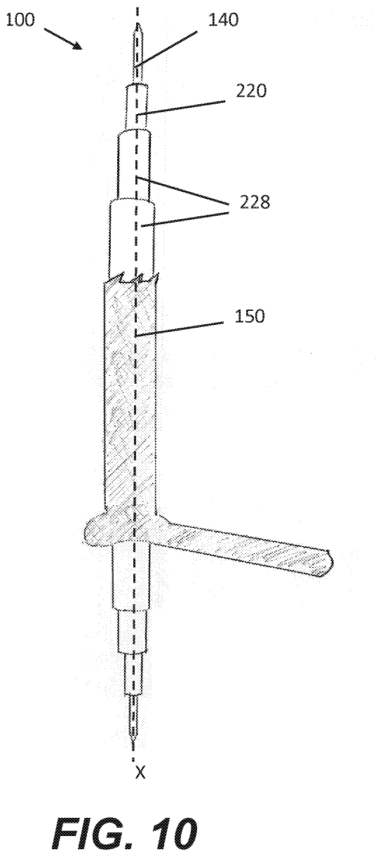

As provided herein, the dimensions of the universal dilator 180 can vary depending on such factors as the size of the patient, the distance between the incision entry point and the surgical target site, and the selection of surgical instruments and devices. Accordingly, the length of the universal dilator 180 as measured from the handle 194 to the distal tip 188 can range from about 15 cm to about 30 cm. For instance, the length of the universal dilator 180 can range from about 20 cm to about 25 cm. In the illustrated example, the universal dilator 180 has a length of about 22 cm. Furthermore, the tubular body 181 of the universal dilator 180 can have an outer diameter that ranges from about 10 mm to about 15 mm. Specifically, the example universal dilator 180 illustrated in FIG. 10 has a tubular body 181 with an outer diameter of about 12 mm. In further examples, the outer diameter of the universal dilator 180 can be about 14 mm, for instance.

### Reamer

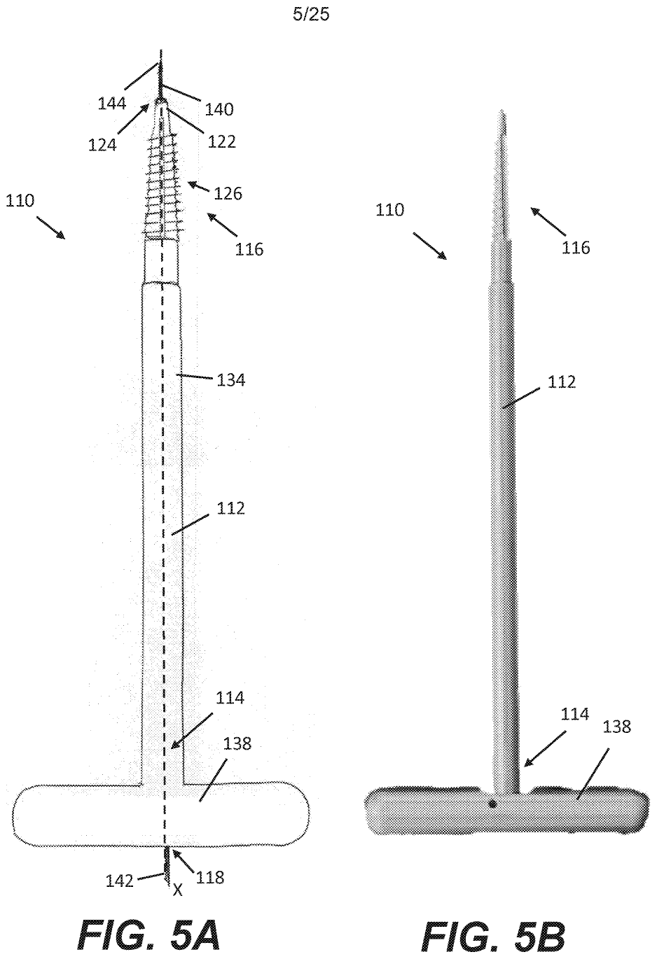

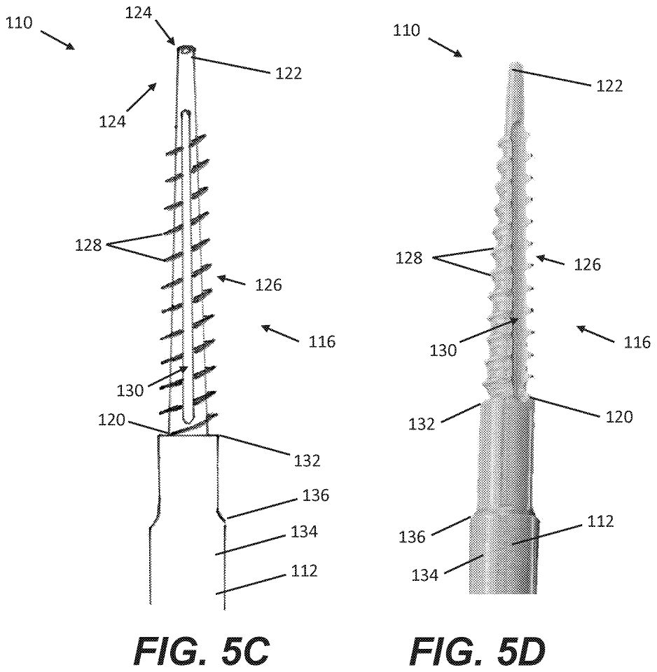

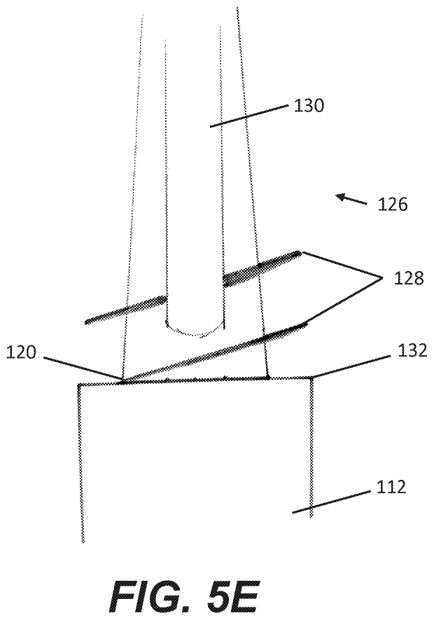

In one aspect, the systems can include one or more reamers 110 for forming a surgical corridor in a patient. FIGS. 5A-9 show reamers 110 according to various examples. Referring to FIG. 5A, an example reamer 110 is shown. The reamer 110 includes a longitudinal body 112 that extends between a proximal portion 114 and a distal portion 116. A central lumen 118 extends longitudinally through the longitudinal body 112. As shown in FIG. 5A (and further shown in FIGS. 5C-5D), the distal portion 116 extends between a base 120 of the distal portion 116 and a distal tip 122 of the distal portion 116. The distal tip 122 defines an opening 124 that is in fluid communication with the central lumen 118. Thus, the central lumen 118 extends through an entire length of the reamer 110. As further described herein, the reamer 110 is thus able to move along the length of a guidance rod 140 and thereby ensuring alignment with a surgical path along which the access corridor will be formed. In the illustrated example, a distal tip 144 of the guidance rod 140 extends from the distal opening 124 of the distal tip 122 of the reamer 110, and a proximal end 142 of the guidance rod 140 extends from the central lumen 118 at the proximal portion 114 of the reamer 110.

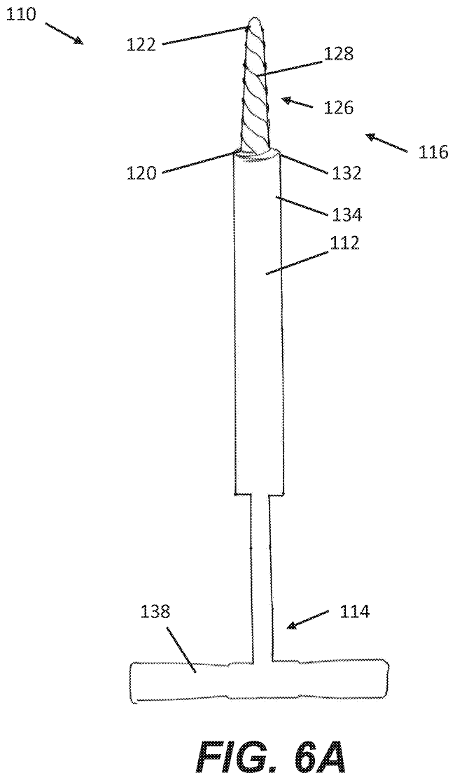

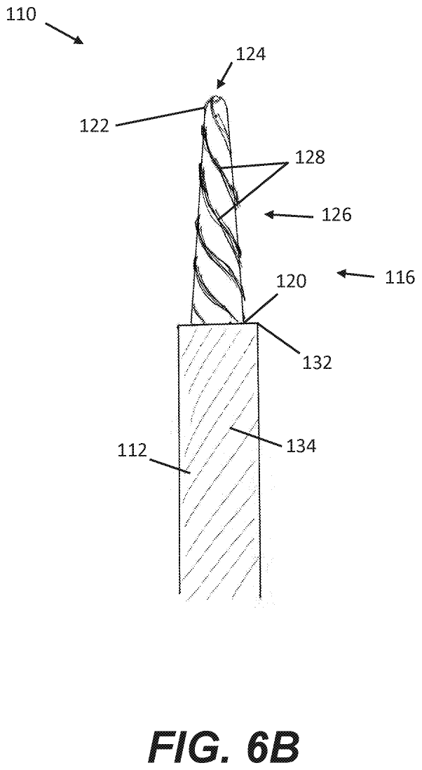

As shown in FIG. 5A and described further in reference to FIGS. 6A-6B, the distal portion 116 includes a tapered region 126 that tapers from the base 120 of the distal portion 116 toward the distal tip 122 of the distal portion 116. Accordingly, the base 120 may also be considered the base of the tapered region 126. At least a portion of the tapered region 126 includes a cutting edge l for removing tissue (e.g., bone) along the surgical path to form the surgical corridor.

In some examples, including the example illustrated in FIG. 5A, the reamer 110 includes a handle 138. As shown, the handle extends orthogonally to the longitudinal body 112 of the reamer 110 and facilitates application of rotational torque about the longitudinal axis X in order to rotate the reamer 110 and advance the reamer 110 along the surgical path. Furthermore, as shown in FIG. 5A, the central lumen 118 of the reamer 110 extends through the handle 138, thereby facilitating translation along the guidance rod 140 extending therethrough.

FIG. 5B shows an example reamer 110. In this example, the handle 138 is formed separately from the reamer 110 and is coupled to the proximal portion 114 by a set screw. In further examples, other means (e.g., complementary threading) may be used to couple the reamer 110 to the handle 138 so long as the reamer 110 and handle 138 remain rotationally fixed relative to the rotational direction of the cutting edge 128 when the reamer 110 progresses along the surgical path. In some examples, the reamer 110 can be operatively connected to a motorized drill that is configured to apply rotational torque to the reamer 110.

Referring now to FIGS. 5C and 5D, detailed views of the distal portion 116 of respective example reamers 110 are shown. Each distal portion 116 includes a tapered region 126 that tapers from the base 120 of the distal portion 116 (and thus also the base of the tapered region 126) toward the distal tip 122 of the distal portion 116. At least a portion of the tapered region 126 includes a cutting edge 128 for removing tissue (e.g., bone) along the surgical path to form the surgical corridor. As shown, the cutting edge 128 extends helically about the tapered region 126. The pitch of cutting edge 128 and the count of helical spirals can vary. The count of rotations of the helical cutting edge 128 about the tapered region 126 can vary depending on such factors as the length and diameter of the tapered region 126, the composition of the tissue to be removed by the 110, and the amount of force available to supply rotational torque to the reamer 110. The example illustrated in FIG. 5C includes a count of about 11 rotations of the helical cutting edge 128 spiraled about the tapered region 126. However, further examples can include other counts of about 5 -15 rotations of the helical cutting edge 128, such as about 12 rotations, about 10 rotations, or about 9 rotations.

In the examples illustrated in FIGS. 5C and 5D, the cutting edge 128 begins at the base 120 of the tapered region 126 and extends toward the distal tip 122. However, the cutting edge 128 is axially spaced from the distal tip 122. In other words, the cutting edge 128 terminates at a distance offset from the opening 124 of the distal tip 122. For example, the cutting edge 128 can be axially spaced from the distal tip 122 by a range of about 5 mm to about 20 mm. Specifically, in the illustrated example, the cutting edge 128 is axially spaced from the distal tip 122 by about 10 mm. In further examples, the cutting edge 128 is axially spaced from the distal tip 122 by about 15 mm. In some examples, the cutting edge 128 can be coated with material to improve cutting efficacy and/or durability. For example, the cutting edge 128 shown in FIG. 5D is steel-coated.

The opening 124 at the distal tip 122 of the reamer 110 is be sized and configured to accommodate translational movement along the guidance rod 140. Accordingly, the opening 124 can range from about 0.5 mm to about 5 mm depending upon the diameter of the guidance rod 140 to be slidably disposed therein. Specifically, the opening 124 of the example reamer 110 illustrated in FIG. 5C is about 2 mm.

The tapered region 126 can have a length ranging from about 25 mm to about 75 mm. For example, the length of the tapered region 126 can range from about 40 mm to about 60 mm. Specifically, in the illustrated example shown in FIG. 5C, the tapered region 126, extending between the base 120 and the distal tip 122 has a length of about 50 mm. In further examples, the tapered region 126 can have a length of about 60 mm, for instance.

In some examples, the base 120 can have a diameter ranging from about 3 mm to about 15 mm. For example, the diameter of the base 120 can range from about 4 mm to about 10 mm. Specifically, as further shown in conjunction with FIG. 5E, the base 120 of the tapered region 126 has a diameter of about 5 mm. As shown in the illustrated example, the longitudinal body 112 extends longitudinally from the base 120.

In some examples, the longitudinal body 112 can include a longitudinally extending barreled portion 134 disposed proximately to the tapered region 126. The barreled portion 134 has a diameter greater than the diameter of the base 120 of the tapered region 126. Furthermore, as shown in the examples illustrated in FIGS. 5C and 5D, the longitudinal body 112 includes a first shoulder 132 and a second shoulder 136 that transition the longitudinal body 112 between the diameter of the base 120 of the tapered region 126 to the diameter of the barreled portion 134. In some examples, the barreled portion 134 can have a diameter ranging from about 5 mm to about 20 mm. For example, the barreled portion 134 can have a diameter ranging from about 10 mm to about 15 mm. Specifically, in the example illustrated in FIGS. 5C and 5E, the diameter of the longitudinal body 112 transitions to a diameter of about 8 mm at the first shoulder 132. This section of the longitudinal body 112 extends about 10 mm longitudinally and then transitions to a diameter of about 11 mm at the second shoulder 136 to define the barreled portion 134. In further examples, the barreled portion 134 can be about 12 mm, for instance. FIG. 5D is a specific example of the reamer 110. As further provided herein, the longitudinal body 112 of the reamer 110 can be sized and configured to slidably translate within an introducer 170.

In some examples the cutting edges 128 can have a diameter that extends beyond the base 120 of the tapered region 126. In the specific example shown in FIG. 5E, the cutting edge 128 extend about 1 mm to about 3 mm from the tapered region 126. In the illustrated example, the cutting edge 128 extends about 1.5 mm from the tapered region 126.

As shown in FIGS. 5C-5E, the tapered region 126 can define a groove 130 extending longitudinally through at least a portion of the cutting edge 128 along an outer surface of at least a portion of the length of the tapered region 126. As further described herein, rotation of the reamer 110 drives the reamer 110 into the bone at the facet joint. The tapered profile of the reamer 110 helps direct bone particulates that are removed by the cutting edge 128 outward away from the treatment site. In particular, bone particulates may be directed toward the groove 130, through which the bone particulates are driven proximally toward the proximal portion 114 of the reamer 110 and away from the treatment site. Advantageously, this reduces the likelihood that bone particulates are accidentally distributed within the incision where such particulates can cause tissue damage. Conversely, some conventional cylindrical reamers can incidentally drive bone toward the incision entry point.

In the example reamers 110 illustrated in FIGS. 5C and 5E, the groove 130 is axially spaced from the distal tip 122 and the base 120 and has a width of about 2 mm. However, in further examples, the groove 130 can range from about 1 mm to about 5 mm, depending on such factors as the diameter of the tapered region 126, the dimensions of the cutting edge 128, and the anticipated volume of tissue to be removed via the groove 130. In FIG. 5D, the groove 130 extends through the base 120 and first shoulder 132. Thus, the groove 130 may be defined according to various configurations so long as it facilitates removal of patient tissue generated upon rotation of the reamer 110.

FIGS. 6A-9 illustrate various additional examples of the reamer 110. Referring to FIGS. 6A-6B, an example reamer 110 is shown in which the cutting edge 128 extends along an entire length of the tapered region 126 between the base 120 and the distal tip 122. In the example illustrated in FIG. 6A, the length of the cutting edge 128 is about 60 mm and the diameter of the barreled portion 134 is about 12 mm. As shown, the transition between the tapered region 126 and the barreled portion 134 is achieved via a single first shoulder 132. Although the specific measurements of the reamer 110 can vary, the barreled portion 134 in the example illustrated in FIGS. 6A-6B has a length of about 14 cm and the reamer has a total length of about 30 cm as measured from the handle 138 to the distal tip 122. FIG. 6B illustrates a detailed view of the distal portion 116 of the reamer 110 shown in FIG. 6A. As shown in FIG. 6B, the opening 124 at the distal tip 122 of the illustrated example has a diameter of about 2.2 mm.

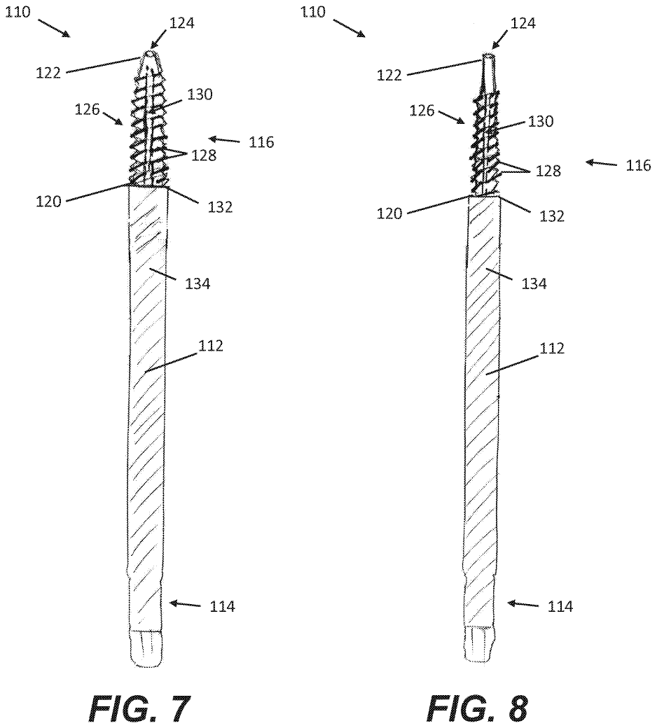

Referring to FIG. 7, an additional example reamer 110 is shown in which the base 120 of the tapered region 126 has a diameter of about 8 mm, the distal tip 122 has a diameter of about 4 mm, the opening 124 has a diameter of about 2 mm, a cutting edge 128 comprising about 10 helical spirals extends from the base 120 toward the distal tip 122, and a groove 130 with a width of about 2 mm is defined through the cutting edge 128 along the tapered region 126. In the illustrated example, the reamer 110 has a total length of about 22.5 cm.

Referring to FIG. 8, an additional example reamer 110 is shown in which the base 120 of the tapered region 126 has a diameter of about 5 mm, the distal tip 122 has a diameter of slightly greater than about 2 mm so as to support an opening 124 having a diameter of about 2 mm, a cutting edge 128 comprising about 9 helical spirals extends from the base 120 toward the distal tip 122, and a groove 130 with a width of about 2 mm is defined through the cutting edge 128 along the tapered region 126. The tapered region 126 extending between the base 120 and the distal tip 122 has a length of about 5.5 mm and the barreled portion 134 of the longitudinal body 112 has a diameter of about 9 mm. In the illustrated example, the reamer 110 has a total length of about 22.5 cm. Specifically, as shown, the cutting edge 128 and groove 130 extend along the tapered region 126 but are spaced apart from the distal tip 122 by about 1.5 mm.

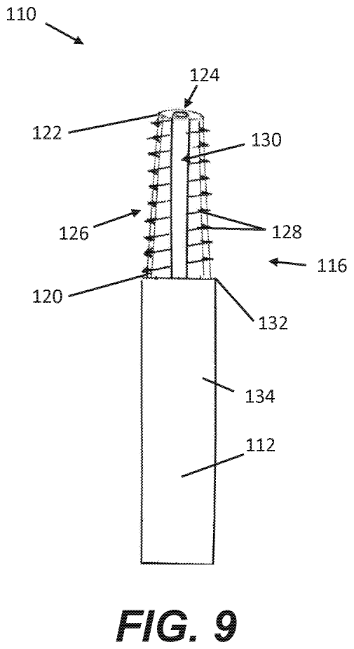

Referring to FIG. 9, an additional example reamer 110 is shown in which the base 120 of the tapered region 126 has a diameter of about 10 mm, the distal tip 122 has a diameter of about 7 mm, the opening 124 has a diameter of about 2 mm, and a cutting edge 128 comprising about 10 helical spirals extends from the base 120 along the entire length of the tapered region 126 to the distal tip 122. Accordingly, the groove 130 is defined through the cutting edge 128 along the entire length of the tapered region 126 to the distal tip 122. In the illustrated example, the groove 130 has a width of about 3 mm, the tapered region 126 extending between the base 120 and the distal tip 122 has a shortened length of about 2.5 mm, and the barreled portion 134 of the longitudinal body 112 has a diameter of about 11 mm. In the illustrated example, the reamer 110 has a total length of about 22.5 cm.

In further examples, specific measurements of the reamer 110 can vary depending on such factors as patient size, target anatomy, and the specifications of subsequent surgical devices and instruments. In fact, multiple sizes of reamers 110 can be used in succession as necessary to gradually create the surgical path toward the target surgical site (e.g., through the facet region into the disc space).

In some examples, the diameter of the base 120 of the tapered region 126 can be about 2 to 5 mm larger than the diameter of the distal tip 122. For instance, in some examples, the base 120 can have a diameter of about 5 mm and the distal tip 122 can have a diameter of about 3 mm. In further examples, the base 120 can have a diameter of about 6 mm and the distal tip 122 can have a diameter of about 4 mm. In further examples, the base 120 can have a diameter of about 7 mm and the distal tip 122 can have a diameter of about 5 mm. In further examples, the base 120 can have a diameter of about 8 mm and the distal tip 122 can have a diameter of about 6 mm. In further examples, the base 120 can have a diameter of 9 mm and the distal tip 122 can have a diameter of about 7 mm. In further examples, the base 120 can have a diameter of about 10 mm and the distal tip 122 can have a diameter of about 8 mm. In further examples, the base 120 can have a diameter of about 11 mm and the distal tip 122 can have a diameter of about 9 mm. In further examples, the base 120 can have a diameter of about 12 mm and the distal tip 122 can have a diameter of about 10 mm. In still further examples, the base 120 can have a diameter of about 11 mm and the distal tip 122 can have a diameter of about 8 mm. In further examples, the base 120 can have a diameter of about 12 mm and the distal tip 122 can have a diameter of about 9 mm.

### Anchoring Dilator

In another aspect, the systems can include one or more dilators for further enlarging the surgical corridor in a patient. FIG. 10 shows an example surgical dilator system 100, including a guidance rod 140, a first tubular dilator 220, additional tubular dilators 228, and an anchoring dilator 150.

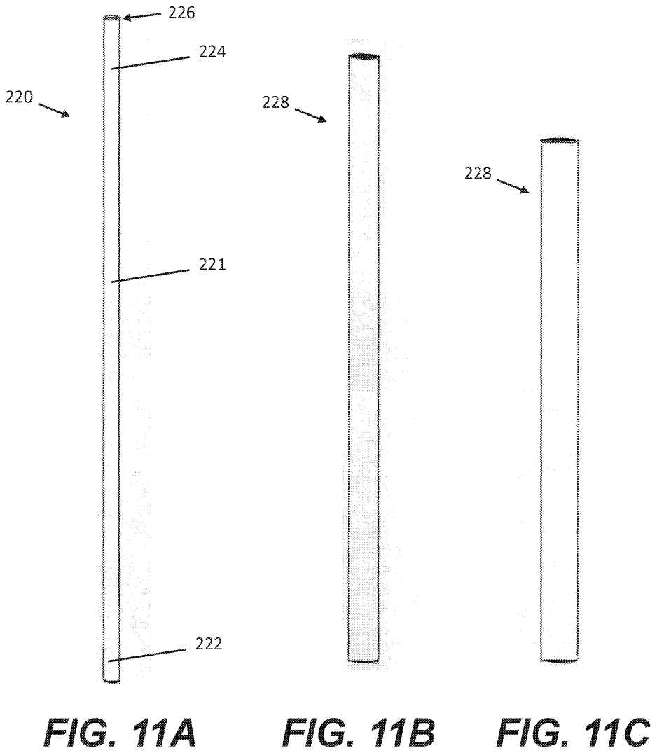

As shown in FIG. 11A, the first tubular dilator 220 includes a tubular body 221 that extends between a proximal portion 222 and a distal portion 224. A central lumen 226 is defined longitudinally between the proximal portion 222 and the distal portion 224 and extends through the entire length of the tubular body 221. As shown in FIG. 10, the central lumen 226 is sized and configured to accommodate translational movement along the guidance rod 140. Accordingly, the diameter of the central lumen 226 can range from about 0.5 mm to about 10 mm depending upon the diameter of the guidance rod 140 to be slidably disposed therein and the extent to which the surgical corridor has already been enlarged by other instruments (e.g., the universal dilator 180 and/or reamer 110). Specifically, the diameter of the central lumen 226 in the example first tubular dilator 220 illustrated in FIG. 10 and FIG. 11A is about 5 mm. Furthermore, the length of the first tubular dilator 220 can range from about 15 cm to about 50 cm. For example, the length of the first tubular dilator 220 can range from about 20 mm to about 30 cm. Specifically, the length of the first tubular dilator 220 illustrated in FIG. 10 and FIG. 11A is about 22 cm.

As further shown in FIG. 10, the example surgical dilator system 100 includes two additional tubular dilators 228 disposed radially outward of the first tubular dilator 220. These additional tubular dilators 228 are illustrated individually, in FIG. 11B and FIG. 11C, respectively. As shown, the additional tubular dilators 228 have central lumens with inner diameters that are greater than the outer diameter of the first tubular dilator 220. Furthermore, the first additional tubular dilator 228 can have lengths that range from about 10 cm to about 30 cm.

Specifically, the first additional tubular dilator 228 shown in FIG. 10 and FIG. 11B has a central lumen with an inner diameter of about 10 mm. This first additional tubular dilator 228 is placed radially outward of the first tubular dilator 220. Furthermore, the first additional tubular dilator 228 shown in FIG. 10 and FIG. 11B has a length of about 20 cm. As shown in FIG. 10, another additional tubular dilator 228 is placed radially outward of the first tubular dilator 220 and the first additional tubular dilator 228. Accordingly, this second additional tubular dilator 228 shown in FIG. 10 and FIG. 11B has a central lumen that is larger than the outer diameters of the first tubular dilator 220 and the first additional tubular dilator 228. In the illustrated example, the central lumen of this second additional tubular dilator 228 has an inner diameter of about 12 mm. Furthermore, the second additional tubular dilator 228 shown in FIG. 10 and FIG. 11C has a length of about 17 cm.

Advantageously, advancement of the first tubular dilator 220 over the guidance rod 140 followed by advancement of sequentially larger additional tubular dilators 228 over the first tubular dilator 220 can gradually enlarge the incision so as to facilitate access for subsequent instruments.

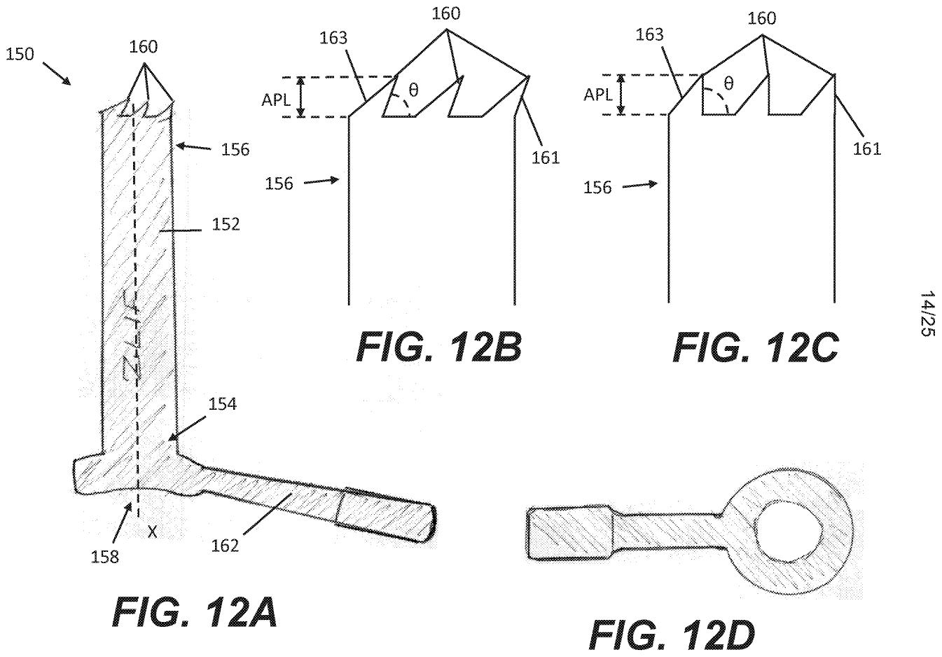

Turning now to FIGS. 12A-12D, an example anchoring dilator 150 is provided. As shown, the anchoring dilator 150 includes a generally tubular longitudinal body 152 extending between a proximal portion 154 and a distal portion 156 along the longitudinal axis X. A central lumen 158 is defined longitudinally within longitudinal body 152 of the anchoring dilator 150 along the longitudinal axis X. As provided herein, the central lumen 158 is sized and configured to facilitate passage of surgical instruments and devices therethrough for formation of the surgical corridor. Once the surgical corridor is formed, the central lumen 158 can provide access to the target surgical site to facilitate delivery of surgical instruments and devices.

The anchoring dilator 150 includes a plurality of serrated protrusions 160 that extend distally from the distal portion 156 of the anchoring dilator 150. As provided herein, the plurality of protrusions 160 are configured to frictionally engage patient tissue, thereby preventing rotation of the anchoring dilator 150 relative to the patient tissue. Specifically, as shown in detailed FIGS. 12B-12D, the plurality of protrusions 160 comprise serrated edges 161 that enable the anchoring dilator 150 to dock on facet joint and resist rotational movement or inadvertent removal of the anchoring dilator 150 from the target surgical site.

In the illustrated examples, the plurality of protrusions 160 are arranged circumferentially about the distal portion 156 (and thus, circumferentially about the longitudinal X axis). In some examples, the plurality of protrusions 160 can be spaced at equidistant intervals.

In some examples, including the examples illustrated in FIGS. 12A-12D, the protrusions 160 of the anchoring dilator 150 have a length (“anchoring dilator protrusion length” or “APL”) ranging from about 1 to about 5 mm. In the illustrated examples, the length of the plurality of protrusions 160 is about 4 mm. In further examples, the length of the plurality of protrusions 160 can be, for instance, about 3 mm or about 1.5 mm.

In some examples, including the examples illustrated in FIGS. 12A-12D, the protrusions 160 extend from the anchoring dilator 150 such that an angle θ is formed between the serrated edges 161 of the plurality of protrusions 160 and the plane extending across the opening of the anchoring dilator 150 at the distal portion 156. As shown in FIG. 12A and in greater detail in FIG. 12B, the angle θ can be an oblique angle. In other words, the plurality of protrusions 160 can extend at an oblique angle relative to the longitudinal axis X. For example, the angle θ can range from about 30° to about 89°. In the illustrated examples, the angle θ is about 60°. In further examples, the angle θ can be about 45°. However, as shown in FIG. 12C, in some examples, the angle θ can be normal, or about 90°. Accordingly, the plurality of protrusions 160 can extend at any angle so long as trailing edges 163 of the protrusions 160 opposite the serrated edges 161 form an oblique angle relative to the plane extending across the opening of the anchoring dilator 150 at the distal portion 156.

Advantageously, the anchoring dilator 150 can thus be inserted into the patient with reduced frictional interference from the plurality of protrusions 160 by gently rotating the anchoring dilator 150 about the longitudinal axis X in a direction opposite the extension direction of the plurality of protrusions 160. For instance, the various example dilators illustrated in FIG. 10 and FIGS. 12A-12D include a plurality of protrusions 160 that extend in a clockwise direction about the longitudinal axis X relative to the direction of insertion. Accordingly, the anchoring dilator 150 can be inserted into the patient with reduced frictional interference with patient tissue by gently rotating the anchoring dilator 150 in a counter-clockwise direction about the longitudinal axis X relative to the direction of insertion. Once the plurality of protrusions 160 reach the surgical target site (e.g., the facet joint), the anchoring dilator 150 can be rotated in the clockwise direction to engage the serrated edges 161 of the plurality of protrusions 160 along a perimeter of the surgical target site so as to anchor the anchoring dilator 150 into the patient's tissue.

As provided herein and shown in FIG. 12D, the central lumen 158 of the anchoring dilator 150 extends longitudinally through the length of the anchoring dilator 150 between the proximal portion 154 and the distal portion 156. The inner diameter of the central lumen 158 can vary in size as appropriate to facilitate passage of surgical instruments and devices therethrough for formation of the surgical corridor. For example, the inner diameter of the central lumen 158 can range from about 10 mm to about 15 mm. In the example anchoring dilators 150 illustrated in FIG. 10 and FIGS. 12A-12D, the inner diameter of the central lumen 158 is 13 mm. Further example anchoring dilators 150 can have central lumens 158 with inner diameters of about 12 mm, about 14 mm, and about 15 mm, for instance. Furthermore, the length of the anchoring dilator 150 can vary depending on factors such as the size of the patient, the distance between the incision entry point and the surgical target site, and the selection of surgical instruments and devices to be delivered via the anchoring dilator 150. In some examples, the length of the anchoring dilator 150 can range from about 5 cm to about 15 cm. For example, the example anchoring dilator 150 illustrated in FIG. 12A has a length of about 9 cm. Further example anchoring dilators 150 can have lengths of about 6 cm, about 12 cm, and about 15 cm, for instance. In some examples, the diameter of the central lumen 158 can vary according to the length of the anchoring dilator 150.

As further shown in FIG. 10, FIG. 12A, and FIG. 12D, the example anchoring dilator 150 includes a handle 162 extending from the proximal portion 154 of the anchoring dilator 150. As provided herein, the handle 162 allows a user to apply rotational force to the anchoring dilator 150 so as to facilitate rotation of the anchoring dilator 150 about the longitudinal axis X. Once the anchoring dilator 150 is anchored into the patient tissue, the handle 162 can then be rotationally fixed in order to stabilize the anchoring dilator 150. For example, the handle 162 can be fixed directly to the patient or to a surgical table via a stabilization arm. In this way, the anchoring dilator 150 serves as an introducer 170 through which subsequent surgical instruments and devices can be moved through the surgical corridor.

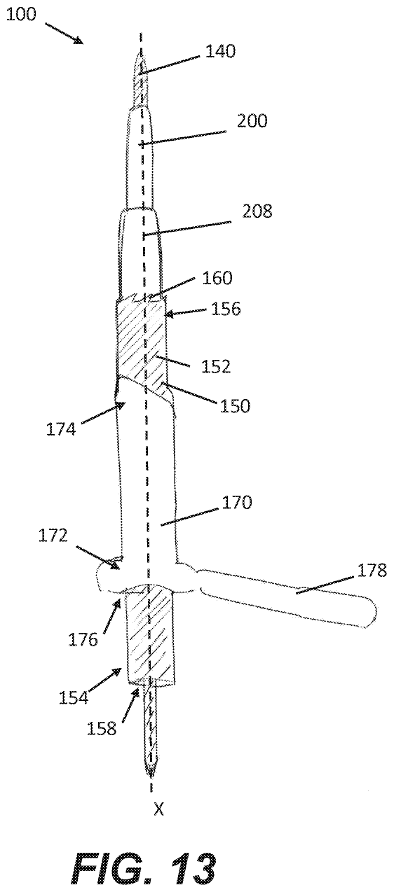

However, as further described herein with reference to FIG. 13, in some example surgical dilator systems 100, the anchoring dilator 150 is used in conjunction with a separately formed introducer 170 that can include an introducer handle 178 not integrally formed with the anchoring dilator 150.

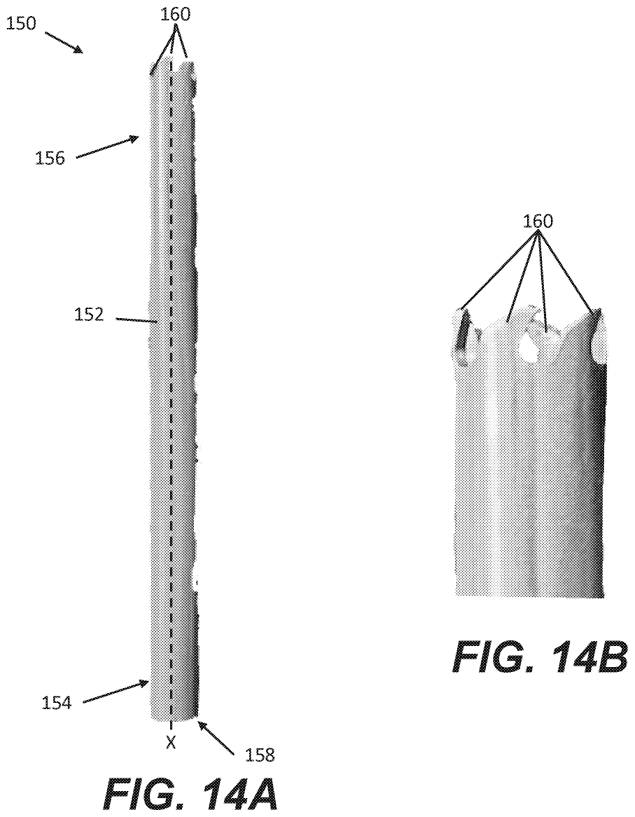

Referring now to FIG. 13, an example surgical dilator system 100 is provided in which an introducer 170 is formed separate from the anchoring dilator 150. Accordingly, the introducer 170 extends between a proximal portion 172 and a distal portion 174 and defines a central lumen 176 extending therethrough. In such examples, subsequent surgical instruments and devices can be moved through the surgical corridor via the central lumen 176 of the introducer 170. As shown in FIG. 13, the central lumen 176 is sized and configured to receive the anchoring dilator 150. In such examples, the introducer 170 includes a handle 178 for fixing rotation of the introducer 170 relative to the longitudinal axis X. For example, the handle 178 can be fixed to the patient directly or to a surgical table via a stabilization arm.

Where the anchoring dilator 150 and introducer 170 are formed separately, the anchoring dilator 150 can be formed as a tubular body that does not include an integrally formed handle 162. FIGS. 14A-14B show such an example anchoring dilator 150. In such examples, the proximal portion 154 of the anchoring dilator 150 can be coupled to a separately formed handle to facilitate rotation of the anchoring dilator 150.

### Joint Stripper

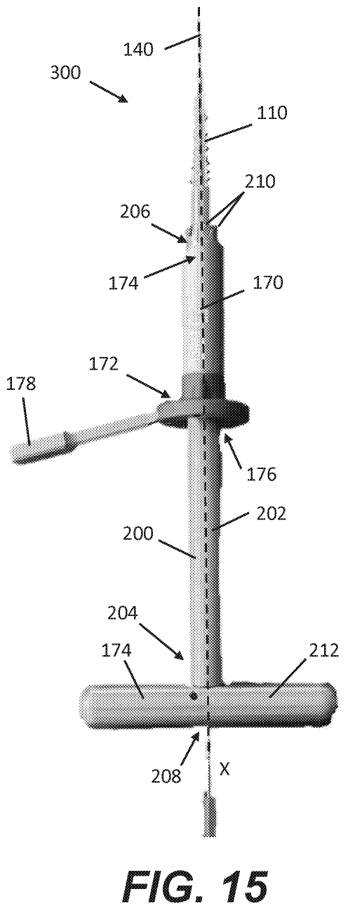

In another aspect, the systems can include a joint stripper 200 for removing soft tissue from a patient's joint. FIG. 15 shows an example surgical reamer system 300 including a guidance rod 140, a reamer 110, a joint stripper 200, and an introducer 170. The joint stripper 200 has a longitudinal body 202 that extends between a proximal portion 204 and a distal portion 206. A central lumen 208 extends longitudinally between the proximal portion 204 and the distal portion 206. As shown in FIG. 15, the central lumen 208 extends the entire length of the joint stripper 200. This facilitates translational movement of the joint stripper 200 over other instruments, such as the guidance rod 140.

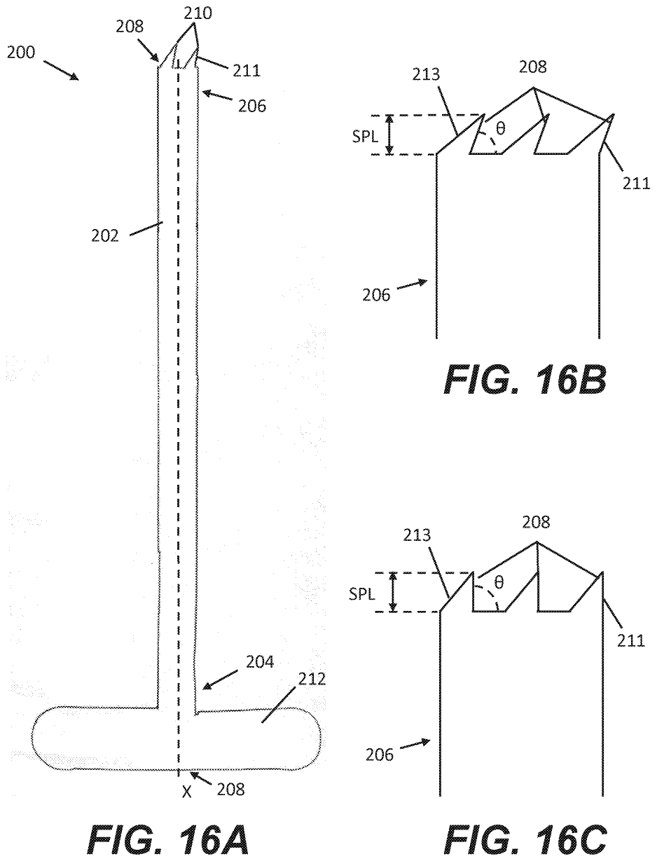

The distal portion 206 of the joint stripper 200 includes a plurality of protrusions 210 that extend distally. As further shown in FIG. 16A, each protrusion 210 has a cutting edge 211 for removing soft tissue from the patient at the surgical target sight (e.g., the facet joint). Advantageously, removing soft tissue from adjacent bones at the surgical target sight improves contact between the adjacent bones, thereby promoting fusion. For example, when performing trans-facet spinal fusion, the joint stripper 200 can be used to remove soft tissue overlaying the facet joint, thereby promoting facet joint fusion and postero-lateral arthrodesis. In some examples, this process can be performed following placement of a fusion-facilitating surgical device into the disc space.

In the illustrated example, the plurality of protrusions 210 are arranged circumferentially about the distal portion 206 (and thus, circumferentially about the longitudinal X axis). In some examples, the plurality of protrusions 210 can be spaced at equidistant intervals.

In some examples, including the examples illustrated in FIG. 15 and FIGS. 16A-16C, the protrusions 210 of the joint stripper 200 have a length (“joint stripper protrusion length” or “SPL”) ranging from about 1 mm to about 5 mm. In the illustrated examples, the length of the plurality of protrusions 210 is about 2 mm.

In some examples, including the examples illustrated in FIGS. 15-16C, the protrusions 210 extend from the joint stripper 200 such that an angle θ is formed between the cutting edges 211 of the plurality of protrusions 210 and the plane extending across the opening of the joint stripper 200 at the distal portion 206. As shown in FIG. 16A and in greater detail in FIG. 16B, the angle θ can be an oblique angle. In other words, the plurality of protrusions 210 can extend at an oblique angle relative to the longitudinal axis X. For example, the angle θ can range from about 30° to about 89°. In the illustrated examples, the angle θ is about 60°. In further examples, the angle θ can be 45°. However, as shown in FIG. 16C, in some examples, the angle θ can be normal, or about 90°. Accordingly, the plurality of protrusions 210 can extend at any angle so long as trailing edges 213 of the protrusions 210 opposite the serrated edges 211 form an oblique angle relative to the plane extending across the opening of the joint stripper 200 at the distal portion 206.

In some examples, the cutting edges 211 can be coated with material to improve cutting efficacy and/or durability. For example, the cutting edges 211 shown in FIGS. 15-16C are steel-coated.

Advantageously, in a manner similar to the anchoring dilator 150, the joint stripper 200 can thus be inserted into the patient with reduced frictional interference from the plurality of protrusions 210 by gently rotating the joint stripper 200 about the longitudinal axis X in a direction opposite the extension direction of the plurality of protrusions 210. For instance, the various example joint strippers 200 illustrated in FIGS. 15-16C include a plurality of protrusions 210 that extend in a clockwise direction about the longitudinal axis X relative to the direction of insertion. Accordingly, the joint stripper 200 can be inserted into the patient with reduced frictional interference with patient tissue by gently rotating the joint stripper 200 in a counter-clockwise direction about the longitudinal axis X relative to the direction of insertion. Once the plurality of protrusions 210 reach the surgical target site (e.g., the facet joint), the joint stripper 200 can be rotated in the clockwise direction to engage the cutting edges 211 of the plurality of protrusions 210 against soft tissue overlaying two adjacent bones so as to remove the soft tissue and promote fusion of the adjacent bones.

As further shown in FIG. 15, and FIG. 16A, the example joint stripper 200 includes a handle 212 extending from the proximal portion 204 of the joint stripper 200. As provided herein, the handle 212 allows a user to apply rotational torque to the joint stripper 200 so as to facilitate rotation of the joint stripper 200 about the longitudinal axis X. In some examples, the joint stripper 200 can be operatively connected to a motorized drill that is configured to apply rotational torque to the joint stripper 200.

As further provided herein, the joint stripper 200 can be sized and configured to slidably translate within an introducer such as introducer 170. In the example illustrated in FIGS. 15 and 16A-16C, the longitudinal body 202 of the joint stripper 200 has a diameter of about 12 mm so as to be slidable within the introducer 170 having an inner diameter of about 13 mm. Furthermore, the illustrated joint stripper 200 has a length of about 22 cm, as measured from the handle 212 to the plurality of protrusions 210. However, as provided herein, the dimensions of the joint stripper 200 can vary depending on such factors as the size of the patient, the distance between the incision entry point and the surgical target site, and the selection of surgical instruments and devices. Thus, depending on the particular surgical workflow implemented, the joint stripper 200 and reamer 110 can be interchangeably inserted into and removed from the anchoring dilator 150/introducer 170.

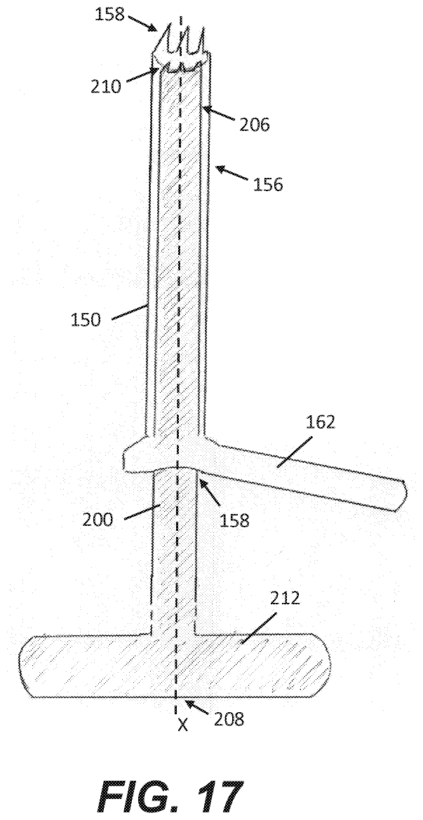

As shown in FIG. 17 and previously described in reference to FIG. 10 and FIGS. 12A-12D, in some examples, the anchoring dilator 150 is configured to also serve as the introducer 170. Accordingly, the joint stripper 200 can be sized and configured to slidably translate within the anchoring dilator 150.

### Disc Preparation Drill

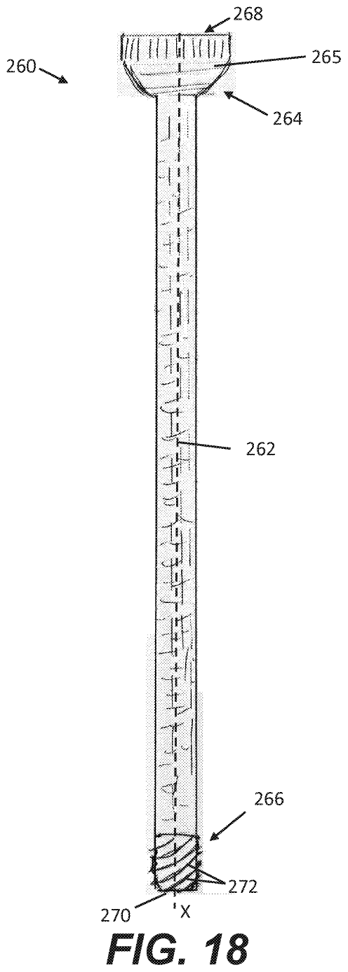

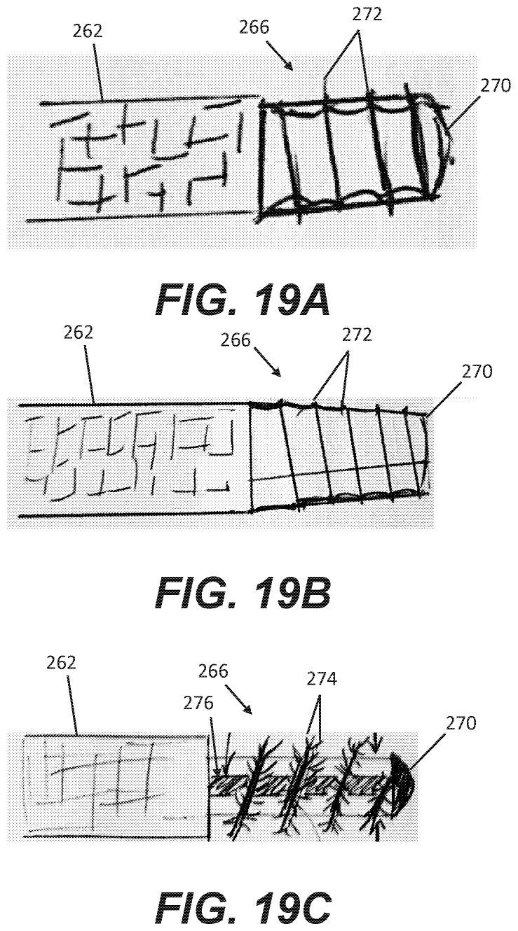

In another aspect, the systems can include a preparation drill 260. FIGS. 18-21C show various exemplary preparation drills 260 for removing tissue from within the disc space so as to provide space for insertion of further surgical instruments and/or implantation of a medical device. Referring to FIG. 18, an example preparation drill 260 is shown. The preparation drill 260 includes a longitudinal body 262 that extends between a proximal portion 264 and a distal portion 266. As shown, the distal portion 266 terminates at a distal tip 270. As shown in FIG. 18, at least a portion of the distal portion 266 includes a cutting edge 272 for removing tissue from the treatment site (e.g., within the disc space). As shown, the cutting edge 272 extends helically about the distal portion 266 and extends to the distal tip 270. In some examples, the cutting edge 272 can be coated with material to improve cutting efficacy and/or durability. For example, the cutting edge 272 shown in FIGS. 18-19B are steel-coated.

A central lumen 268 extends longitudinally through at least a portion of the longitudinal body 262 and defines an opening at the proximal portion 264 of the preparation drill 260. In the illustrated example, the distal tip 270 does not extend all the way through the distal tip 270. In other words, the distal tip 270 in the illustrated example does not include an opening.

As further shown in FIG. 18, the proximal portion 264 of the preparation drill 260 includes a handle 265. As shown the opening of the central lumen 268 is defined by the handle 265. As provided herein, the handle 265 can be used to move the preparation drill 260 axially along the longitudinal axis X and rotate the preparation drill 260 about the longitudinal axis X. In some examples, the preparation drill 260 can be operatively connected to a motorized drill that is configured to apply rotational torque to the preparation drill 260.

In some examples, the diameter of the longitudinal body 262 can range from about 3 mm to about 15 mm. In some examples, the diameter of the longitudinal body 262 can range from about 5 mm to about 9 mm. In some specific examples, the diameter of the longitudinal body 262 can 5 mm, or 7 mm, or 9 mm, for instance. In some examples, the diameter of the distal tip 270 can range from about 2 mm to about 12 mm. In the illustrated example, the longitudinal body 262 has a diameter of about 9 mm and the distal tip 270 has a diameter of about 7 mm. Furthermore, in some examples the preparation drill 260 has a total length that can range from about 20 cm to about 40 cm. For instance, the example preparation drill 260 illustrated in FIG. 18 has a total length of about 28 cm. However, as provided herein, the dimensions of the preparation drill 260 can vary depending on such factors as the size of the patient, the distance between the incision entry point and the surgical target site, and the selection of surgical instruments and devices.

FIGS. 19A-19C show distal portions 266 of various exemplary preparation drills 260. In FIG. 19A, the exemplary cutting edge 272 extends radially from the longitudinal body distal portion 266 to define a cutting diameter that is approximately the same as the diameter of the longitudinal body 262. In other words, the cutting edge 272 defines a cylindrically-shaped cutting region.

In FIG. 19B, the exemplary distal portion 266 tapers toward the distal tip 270. Accordingly, the cutting region defined by the cutting edge 272 also tapers toward the distal tip 270. In some examples, the length of the tapered distal portion 266 can range from about 1 mm to about 5 mm. In the illustrated example, the length of the tapered distal portion 266 has a length of about 1.5 mm.

In FIG. 19C, the exemplary cutting edge 272 can be in the form of a plurality of radially extending bristles 274 that wrap helically around the distal portion 266. As shown in FIG. 19C, the plurality of bristles 274 can extend in multiple directions. In some examples, the plurality of bristles 274 have a lengths that can range from about 1 mm to about 5 mm. In the illustrated example, the plurality of bristles 274 have lengths of about 2 mm. In some examples, the plurality of bristles 274 can be formed from a hard material such as metal to improve cutting efficiency and/or durability. In the exemplary preparation drill 260 illustrated in FIG. 19C, the distal portion 266 from which the plurality of bristles 274 extends has a reduced diameter relative to the longitudinal body 262. In some examples, the diameter of the distal portion 266 from which the plurality of bristles 274 extends can range from about 2 mm to about 10 mm. In the illustrated example, the distal portion 266 from which the plurality of bristles 274 extends can has a diameter of about 5 mm. Furthermore, the distal portion 266 in the illustrated example has a constant diameter such that the plurality of bristles 274 define a cylindrically-shaped cutting region.

In some examples, such as the example preparation drill 260 illustrated in FIG. 19C, the distal portion 266 defines a groove 276 extending longitudinally through at least a portion of the cutting edge 272. Rotation of the preparation drill 260 drives the cutting edge 272 into tissue within the treatment area. In some instances, action of the cutting edge 272 may cause tissue particulates to develop. It is contemplated herein that the groove 276 may help direct such bone particulates proximally away from the treatment site. Advantageously, this reduces the likelihood that tissue particulates are accidentally distributed within the incision where such particulates can cause tissue damage.

In some examples, the cutting edge 272 formed on the distal portion 266 are integral with the longitudinal body 262 of the preparation drill 260. In other examples, the cutting edge 272 is formed as a separate drill bit that is couplable to the longitudinal body 262.

As further provided herein, the longitudinal body 262 of the preparation drill 260 is formed from a flexible material. For example, the longitudinal body 262 can be made of a Flex-Metric® structure. Advantageously, this allows the longitudinal body 262 to deflect (e.g., curve) relative to the longitudinal axis X, thereby permitting flexion of the distal portion 266 and the cutting edge 272 positioned thereon. Furthermore, the flexible character of the longitudinal body 262 enables rotation of the preparation drill 260 even when the longitudinal body 262 is deflected (e.g., curved) relative to the longitudinal axis X. In some examples, the cutting edge 272 can thus be manipulated within a treatment area.

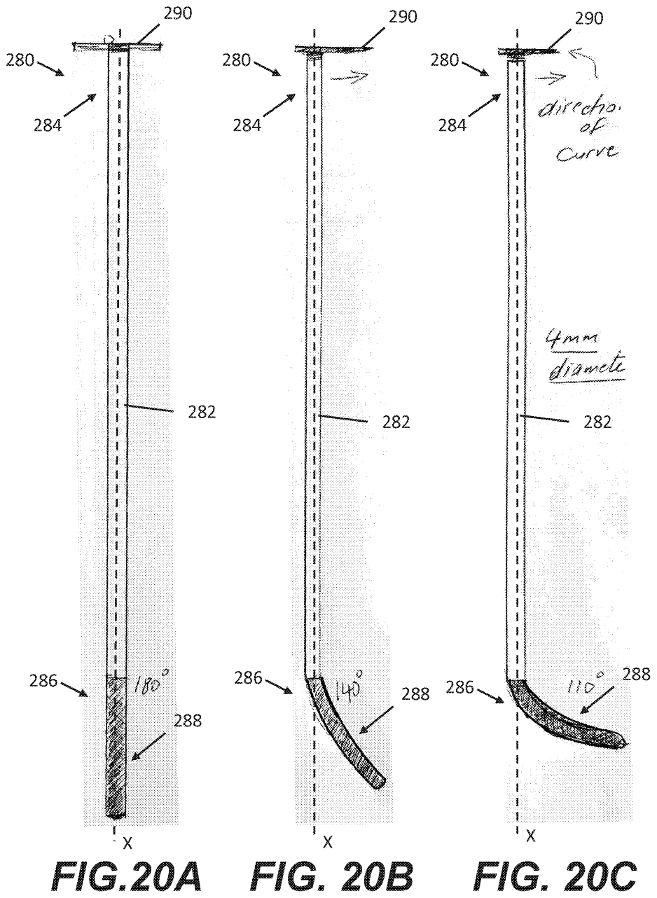

FIGS. 20A-20C relate to an articulating stylet 280 that is configured for insertion into the central lumen 268 of the preparation drill 260. As shown, the articulating stylet 280 has a longitudinal body 282 that extends between a proximal portion 284 and a distal portion 286. Notably, the distal portion 286 includes an articulating region 288 that is configured to deflect relative to the longitudinal axis X. Furthermore, the proximal portion 284 of the articulating stylet 280 includes a handle 290 that is configured to induce deflection of the articulating region 288 relative to the longitudinal axis X.

FIG. 20A shows an example articulating stylet 280 before the articulating region 288 is deflected. Accordingly, the distal portion 286 of the articulating stylet 280, including the articulating region 288, is axially aligned with the longitudinal axis X. As shown in FIG. 20B, manipulation of the handle 290 facilitates deflection of the articulating region 288 relative to the longitudinal axis X. Specifically, FIG. 20B shows the articulating region 288 deflected at a moderate angle of 40° relative to the longitudinal axis X. As shown in FIG. 20C, further manipulation of the handle 290 can facilitate further deflection of the articulating region 288 relative to the longitudinal axis X. Specifically, FIG. 20B shows the articulating region 288 deflected at a sharp angle of 70° relative to the longitudinal axis X.

Accordingly, the articulating stylet 280 can have an outer diameter that is sized and configured to translate within the central lumen 268 of the preparation drill 260. Conversely, the central lumen 268 of the preparation drill 260 has an inner diameter that is sized and configured to allow translation of the articulating stylet 280 within the central lumen 268.

### Example Surgical Technique

It is contemplated that the systems and method provided herein can be utilized to achieve various surgical objectives. As previously described, one particular use for the systems and methods provided herein is the performance of minimally invasive spinal surgery via a trans-facet approach into a zygapophysial joint. In some examples, the trans-facet approach may be used to form an access corridor through the facet joint (i.e., a “trans-facet access corridor”) in order to facilitate intervention of conditions including stenosis, herniated discs, fractures, spinal deformities, spondylolisthesis, or degenerative disc diseases. In some instances, spinal fusion may be indicated.

### Trans-Facet Lumbar Interbody Fusion

Provided herein is one example of the present approach in the context of interbody lumbar fusion. However, it is contemplated herein that the approach can be performed in the context of any number of other spinal surgeries, including fusion of thoracic or cervical vertebrae, as well as surgeries that do not involve fusion, such as spinal decompression surgery. Accordingly, the surgical instruments provided herein can vary in size as appropriate for a particular surgical application. For example, surgical instruments having smaller dimensions may be appropriate for patients with smaller anatomical features, such as pediatric patients, whereas surgical instruments having relatively larger dimensions may be appropriate for patients with comparatively larger anatomical features. Furthermore, although various surgical instruments provided herein can optionally include handles, each surgical instrument can instead be manipulated via a robotic surgical guidance system.

In some examples, presurgical planning can be conducted to plan the surgical approach and thus the formation of the surgical corridor using the instruments described herein. In some examples, a robotic navigation system and/or live fluoroscopy can be implemented to verify the location of the surgical target site, mark the incision entry point, and guide the approach toward the target surgical site (e.g., at the midline of the facet joint).

For example, under image guidance such as fluoroscopy, a small incision can be made at the marked incision entry point and the access cannula 230 can be introduced by piercing the patient's skin with the pointed distal tip 252 of the stylet 250 disposed within the access cannula 230. This creates an incision in the patient's skin that is approximately the diameter of the access cannula 230. For example, the incision can be about 1.5 cm wide.

The access cannula 230 can then be advanced until contacts the facet joint. A mallet can optionally be used to seat the access cannula 230 into the facet joint. In some examples, an access cannula 230 such as the one illustrated in FIGS. 22A-22D is used such that the access cannula 230 is advanced into contact with the facet joint and rotated so that the threaded portion 242 engages the facet joint, coupling the access cannula 230 to the facet joint. For example, the access cannula 230 can be rotated into engagement with the facet joint so that the access cannula 230 is advanced into the facet joint and/or disc space. The stylet 250 can then be removed from the access cannula 230 and the guidance rod 140 can be inserted into the access cannula 230 and advanced along the surgical path. In some examples, an electric drill can be used to drive the guidance rod 140 along the surgical path toward the target site. Image guidance such as fluoroscopy can be used to further guide placement of the guidance rod 140 and ensure the distal tip 144 of the guidance rod 140 contacts the facet joint. For example, the guidance rod 140 can be seated within tissue of the facet joint superficial relative to the treatment location interior to the facet joint. Thus, as described herein, the guidance rod 140 defines the surgical path that will guide the movement of subsequent surgical instruments and thereby form the surgical corridor.

With the guidance rod 140 in place, the access cannula 230 can be removed. For example, in some implementations, the access cannula 230 can be rotated out of engagement with the facet joint and removed. In some examples, the access cannula 230 can remain in place during the procedure and the dilators 150, 180, 220, 228, reamer 110, introducer 170, drill 260, stylet 280, joint stripper 200, and/or other instruments described herein for creating and maintaining the surgical corridor and/or delivering a medical device/therapy to the treatment site can be advanced through the central lumen of the access cannula 230.

In order to continue the incision deeper through the fascia, the universal dilator 180 can be advanced over the guidance rod 140 into the incision and moved toward the target site. The tapered region 186 at the distal portion 184 of the universal dilator 180 creates an opening in the fascia. The universal dilator 180 can then be removed.

A reamer 110 can then be advanced over the guidance rod 140 along the surgical path to further expand the surgical corridor by removing tissue (e.g., bone). Rotation of the reamer 110 drives the reamer 110 into the bone at the facet joint. The tapered profile of the reamer 110 helps direct bone particulates that are removed by the cutting edge 128 outward away from the incision site. In particular, bone particulates may be directed toward the groove 130 and driven proximally toward the proximal portion 114 of the reamer 110 away from the treatment site and out of the incision. Depending on the requirements of the surgical procedure, additional tapered reamers can be utilized to further remove bone. For example, a series of reamers 110, each with tapered regions 126 having diameters larger than the previous, can be sequentially driven into and rotated within the surgical corridor, thereby gradually enlarging the surgical corridor as needed.

With the opening formed in the fascia, the first tubular dilator 220 and one or more additional tubular dilators 228 can then be advanced over the guidance rod and moved toward the target site. This can include placing a series of increasingly larger diameter tubular dilators 220, 228 sequentially, thereby gradually enlarging the surgical corridor to facilitate access for subsequent instruments. In the context of a trans-facet approach, the tubular dilators 220, 228 spread the patient's paraspinal muscle fibers.

With the first tubular dilator 220 and one or more additional tubular dilators 228 in place, anchoring dilator 150 can be advanced over the tubular dilators 220, 228. As provided herein, the anchoring dilator 150 can be advanced by generally rotating the anchoring dilator 150 in a direction opposite the serrated edges 161 on protrusions 160 such that the serrated edges 161 do not catch on tissue during placement (e.g., counter-clockwise direction about the longitudinal axis X). Once the plurality of protrusions 160 reach the surgical target site (e.g., the facet joint), the anchoring dilator 150 can then be anchored in place by rotating the anchoring dilator 150 in a direction that advances the serrated edges 161 into the patient's tissue (e.g., clockwise). Thus, the serrated edges 161 provide a secure grip to the facet joint that resists counter-rotation of the anchoring dilator 150 relative to the facet joint.

In some examples, rotation of the anchoring dilator 150 can be achieved by applying torque to the handle 162 of the anchoring dilator 150. In some examples, a user can tap the end of the proximal portion 154 of the anchoring dilator 150 with a mallet or hammer to promote stable anchoring. In some examples, the anchoring dilator 150 also serves as an introducer 170 through which subsequent instruments can be advanced toward the treatment site. In such examples, the anchoring dilator 150 includes a handle 162 for rotationally fixing the anchoring dilator 150 directly to the patient or to the surgical table via a stabilization arm.

In this way, the anchoring dilator 150 can itself serve as an introducer 170 through which subsequent surgical instruments and devices can be moved through the surgical corridor.

In further examples, a separate introducer 170 with a handle 178 can be advanced over the anchoring dilator 150 along the surgical corridor and coupled (e.g., rotationally coupled) to the anchoring dilator 150.

Once the anchoring dilator 150/introducer 170 is anchored into the facet joint, the tubular dilators 220, 228 can be removed. The guidance rod 140 can optionally be left in place to facilitate advancement of additional instruments along the surgical path.

In some examples, the integrity of the surgical corridor and/or progress of the surgical corridor can then be confirmed with direct vision using intraoperative microscopy or endoscopy. If necessary, subsequent instruments can be inserted into the anchoring dilator 150/introducer 170 to further clean or prepare the treatment site (e.g. curets). In further examples, where the surgical corridor facilitates access to the disc space of a facet joint, disc material from the disc space can be removed via one or more instruments advanced through the surgical corridor, including any one or more of the instruments described herein.

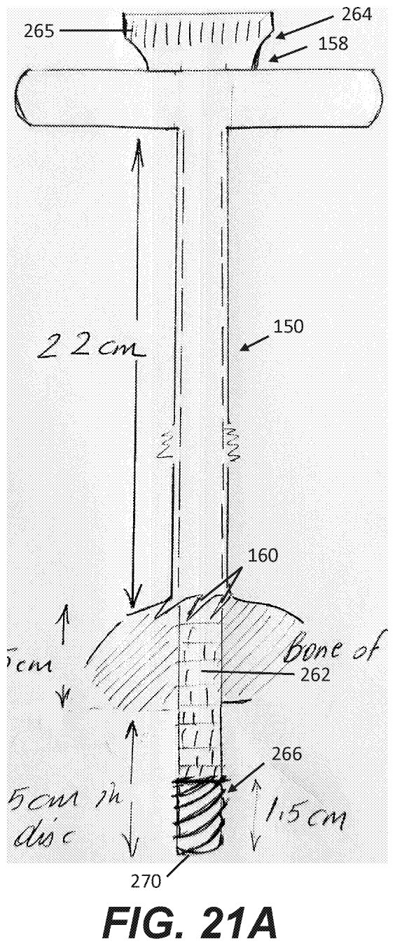

In some examples, a preparation drill 260 can be utilized to further remove tissue from the treatment site. FIGS. 21A-21B show an example preparation drill 260 in use in combination with an example anchoring dilator 150. As shown, the plurality of protrusions 160 of the anchoring dilator 150 are anchored into a facet joint and the surgical corridor is at least partially defined through the central lumen 158. As illustrated in FIG. 21A, the preparation drill 260 can be inserted into the central lumen 158 of the anchoring dilator 150 and advanced into the treatment site. In the illustrated example, the treatment site includes the disc space of a facet joint. In some examples, the distal tip 270 of the preparation drill 260 can be inserted from a range from about 2 mm to about 8 mm into the disc space of the facet joint. In the illustrated example, the distal tip 270 of the preparation drill 260 is inserted about 5 cm into the disc space of the facet joint.

The handle 265 of the preparation drill 260 can then be rotated in order to rotate the distal portion 266 and the cutting edge 272 disposed on the cutting edge 272, thereby removing tissue from the treatment site (e.g., disc space). The depth of insertion of the distal tip 270 into the treatment site (e.g., disc space) can be approximated using the position of the handle 265 and/or markings on the longitudinal body 262 of the preparation drill 260 for reference.

Once the preparation drill 260 is inserted into the treatment site (e.g., the disc space), the articulating stylet 280 can be inserted into the central lumen 268 of the preparation drill 260. As described herein, manipulation of the handle 290 facilitates deflection of the articulating region 288 of the articulating stylet 280 relative to the longitudinal axis X. Accordingly, as shown, in FIG. 21B, when the articulating stylet 280 is disposed within the central lumen 268, the handle 290 can be manipulated to facilitate deflection of the flexible longitudinal body 262 (and distal portion 266) of the preparation drill 260 relative to the longitudinal axis X. Specifically, FIG. 20B shows the longitudinal body 262 and distal portion 266 of the preparation drill 260 deflecting relative to the longitudinal axis X and thus moving laterally within the treatment region (e.g., facet joint). Accordingly, the cutting edge 272 disposed on the distal portion 266 can be maneuvered within a volume of the treatment site defined by the extent to which the distal portion 266 of the preparation drill 260 can be deflected (e.g., curved) by the articulating region 288 of the articulating stylet 280. In some examples, the distal portion 266 of the preparation drill 260 can be deflected from a range of 0° to 90° relative to the longitudinal axis X. In the illustrated example, the distal portion 266 is deflected (e.g., curved) at a moderate angle of 40° relative to the longitudinal axis X.

Furthermore, as shown in FIG. 21B, the distal portion 266 of the preparation drill 260 can be rotated while in various deflected (e.g., curved) states. Advantageously, the preparation drill 260 is thus able to engage tissue within a large volume of the treatment stie (e.g., disc material from the disc space), including at locations radially displaced from the surgical corridor as defined by the central lumen 158 of the anchoring dilator 150.

In some examples, the preparation drill 260 is thus able to create a vacancy in the treatment site into which additional surgical instruments, medical devices, graft materials, or tissue implants can be placed.

Once the surgical corridor is formed, a surgical procedure, such as spinal fusion, can be performed that can include placement of a medical device and/or graft material into the disc space. In further examples, a tissue implant, such as an intervertebral disc, can be inserted into the disc space to help restore function of the injured vertebrae.

Where the surgical procedure involves spinal fusion, it is desirable to promote fusion of the facet joint. However, the facet joint includes a joint capsule of soft tissue that can interfere with bone fusion. Accordingly, a joint stripper 200 can be inserted into the anchoring dilator 150/introducer 170 and rotated to remove soft tissue at the joint capsule and expose the bone of the facet joint. Specifically, as provided herein, the joint stripper 200 can be advanced by generally rotating the joint stripper 200 in a direction opposite the cutting edges 211 on the plurality of protrusions 210 such that the cutting edges 211 do not catch on tissue during placement (e.g., counter-clockwise direction about the longitudinal axis X). Once the plurality of protrusions 210 reach the surgical target site (e.g., the facet joint), the joint stripper 200 can be rotated in a direction that engages the cutting edges 211 of the plurality of protrusions 210 against soft tissue overlaying two adjacent bones so as to remove the soft tissue and promote fusion of the adjacent bones (e.g., clockwise). Advantageously, removing soft tissue from the joint provides surfaces for adjacent regions of bone to engage in direct contact, thereby promoting fusion of the joint.

### Additional Examples of the Surgical Corridor

As described above, the trans-facet approach provided herein can include forming an access corridor through the facet joint between the superior articular process of the vertebra below, and the inferior articular process of a vertebra above the disc space. The trans-facet approach can be performed at any level in the spine. In specific examples, the access corridor according to the trans-facet approach described herein can be formed in any vertebra from the second cervical vertebra (C2) to the sacrum (S1).

In some implementations, the access corridor extends along a generally straight trajectory traversing the inside of the facet joint without exposing surrounding neural elements, such as the exiting nerve roots and the thecal sac. Advantageously, the trans-facet access corridor is separated from the thecal sac and the exiting nerve roots by remaining bony elements of the superior and inferior articular processes that are preserved during and after formation of the trans-facet access corridor. Moreover, the trans-facet access corridor can be separated from the thecal sac medially by the ligamentum flavum, and separated from the exiting nerve root laterally by the facet joint capsule.

The articulation between one superior articular process of the vertebra below the disc space and the inferior articular process of the vertebra above the disc space have different orientations in different regions of the spine. In the lumbar region, the articulation is generally vertically aligned along the sagittal plane. However, in the cervical and thoracic regions of the spinal column, the orientation of the articulation favors the coronal plane. Furthermore, the sizes of the facet joints vary along the spinal column, with facet joints positioned within the cervical and thoracic regions being relatively smaller than those positioned in the lumbar and sacral regions. These variations in the orientation and relative sizes of the facet joints correspond to variations in the trajectory and orientation of the trans-facet access corridor. Furthermore, these variations in the sizes of the facet joints may inform selection of varieties of the instruments disclosed herein that are larger or smaller as appropriate in the context of the facet joint targeted for operation.

Furthermore, while in some examples the entry point of the trans-facet access corridor into the joint is located midway between the inferior articular process and superior articular process, it is contemplated herein that the entry point can be located closer to one or the other of the inferior articular process or superior articular process. For example, in some implementations, preoperative planning informed by the above-referenced anatomical factors can be implemented to determine the percentage of drilling through one articular process versus the other. Such off-midline entry points are consistent with the approach provided herein, so long as aspects of both the inferior articular process and superior articular process remain on either side of the access point. Specifically, in some examples, the trans-facet access corridor is surrounded by bone on all sides, with the possible exception of the planar region formed at the synovial joint interface of the inferior articular process and superior articular process themselves. Therefore, in various examples, the approach provided herein does not include performing a total facetectomy or even a partial (e.g., medial or lateral) facetectomy.

For example, in some examples, the entry point may be offset from a midpoint between the inferior articular process and superior articular process such that a range from 1% to 99% of the material removed to form the access corridor is removed from one of the inferior articular process or the superior articular process. For example, in some examples, the access point can be disposed such that 10% of the material removed to form the access corridor is removed from one of the inferior articular process or the superior articular process and 90% of the material is removed from the other of the inferior articular process or the superior articular process. In some examples, the access point can be disposed such that 20% of the material removed to form the access corridor is removed from one of the inferior articular process or the superior articular process and 80% of the material is removed from the other of the inferior articular process or the superior articular process. In some examples, the access point can be disposed such that 30% of the material removed to form the access corridor is removed from one of the inferior articular process or the superior articular process and 70% of the material is removed from the other of the inferior articular process or the superior articular process. In some examples, the access point can be disposed such that 40% of the material removed to form the access corridor is removed from one of the inferior articular process or the superior articular process and 60% of the material is removed from the other of the inferior articular process or the superior articular process. Furthermore, as described herein, in some examples, the access point can be disposed midway between the inferior articular process and the superior articular process such that 50% of the material removed to form the access corridor is removed from one of the inferior articular process or the superior articular process and 50% of the material is removed from the other of the inferior articular process or the superior articular process. As described herein, any ratio is consistent with this disclosure so long as the access corridor is surrounded degrees by bony elements of the superior and/or inferior articular processes (with the possible exception of the planar region formed at the synovial joint interface of the inferior articular process and superior articular process themselves).

Furthermore, while in some examples, forming the trans-facet access corridor involves accessing the disc space itself, in other examples, the trans-facet access corridor does not extend into the disc space. In such examples, a device such as a medical device such as a cage, a flexible pouch, and/or a graft material can be placed within the trans-facet access corridor but not into the disc space. In such cases, the medical device or graft material may be any of the interbody devices described herein or may be another medical device or material not suitable for implantation within the disc space.

Although specific examples of the disclosure have been described, one of ordinary skill in the art will recognize that numerous other modifications and alternative examples are within the scope of the disclosure. For example, while various illustrative examples and structures have been described in accordance with examples of the disclosure, one of ordinary skill in the art will appreciate that numerous other modifications to the illustrative examples and structures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments.

### Exemplary Aspects

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: A surgical reamer system comprising: a reamer for forming a surgical corridor in a patient, the reamer comprising a longitudinal body extending between a proximal portion and a distal portion and defining a central lumen extending longitudinally therethrough, wherein the distal portion comprises a tapered region that extends between a base and a distal tip and tapers toward the distal tip, wherein at least a portion of the tapered region comprises a cutting edge for removing bone along the surgical corridor, wherein the distal tip defines an opening in fluid communication with the central lumen.

Example 2: A surgical reamer system according to any example herein, particularly example 1, wherein the cutting edge of the reamer extends helically about the tapered region.

Example 3: A surgical reamer system according to any example herein, particularly examples 1-2, wherein the cutting edge of the reamer extends along an entire length of the tapered region.

Example 4: A surgical reamer system according to any example herein, particularly examples 1-3, wherein the cutting edge of the reamer is axially spaced from the distal tip.

Example 5: A surgical reamer system according to any example herein, particularly examples 1-4, wherein the cutting edge of the reamer is steel-coated.

Example 6: A surgical reamer system according to any example herein, particularly examples 1-5, wherein the distal portion of the reamer defines a groove extending longitudinally along an outer surface of the distal portion.

Example 7: A surgical reamer system according to any example herein, particularly example 6, wherein the groove extends through at least a portion of the cutting edge.

Example 8: A surgical reamer system according to any example herein, particularly example 7, wherein the groove extends to the base of the tapered region.

Example 9: A surgical reamer system according to any example herein, particularly examples 1-8, wherein the base of the tapered region has a diameter that is 3 mm larger than a diameter of the distal tip.

Example 10: A surgical reamer system according to any example herein, particularly examples 1-9, wherein a distance between the base of tapered region and the distal tip of the distal portion has a length of 6 cm.

Example 11: A surgical reamer system according to any example herein, particularly examples 1-10, wherein the longitudinal body of the reamer comprises a barreled portion having a diameter greater than a diameter of the base of the tapered region.

Example 12: A surgical reamer system according to any example herein, particularly examples 1-11, wherein the reamer further comprises a handle for rotating the reamer about a longitudinal axis:

Example 13: A surgical reamer system according to any example herein, particularly example 1, further comprising: a joint stripper for removing soft tissue from a patient's joint, the joint stripper comprising a longitudinal body extending between a proximal portion and a distal portion and defining a central lumen extending longitudinally therethrough, wherein the distal portion of the joint stripper comprises a plurality of distally extending protrusions, each protrusion comprising a cutting edge for removing soft tissue from the patient's joint.

Example 14: A surgical reamer system according to any example herein, particularly example 13, wherein the joint stripper further comprises a handle for rotating the joint stripper about a longitudinal axis.

Example 15: A surgical reamer system according to any example herein, particularly examples 13-14, wherein the joint stripper is disposable within an anchoring dilator, the anchoring dilator comprising: a proximal portion and a distal portion and defining a central lumen extending longitudinally therethrough, wherein the distal portion comprises a plurality of distally extending protrusions for frictionally engaging patient tissue.

Example 16: A surgical dilator system for enlarging a corridor in a patient, the surgical dilator system comprising: a guidance rod extending between a proximal end and a tapered distal tip; a first tubular dilator extending between a proximal portion and a distal portion and defining a central lumen extending longitudinally therethrough, wherein the central lumen is sized and configured to receive the guidance rod; and an anchoring dilator extending between a proximal portion and a distal portion and defining a central lumen extending longitudinally therethrough, wherein the distal portion comprises a plurality of distally extending protrusions for frictionally engaging patient tissue, wherein the central lumen of the anchoring dilator is sized and configured to receive the guidance rod and first tubular dilator.

Example 17: A surgical dilator system according to any example herein, particularly example 16, wherein the surgical system further comprises one or more additional tubular dilators disposed radially outward of the first tubular dilator.

Example 18: A surgical dilator system according to any example herein, particularly examples 16-17, wherein the plurality of distally extending protrusions comprise a plurality of serrated edges.

Example 19: A surgical dilator system according to any example herein, particularly examples 16-18, wherein the plurality of distally extending protrusions extend circumferentially about the distal portion of the anchoring dilator.

Example 20: A surgical dilator system according to any example herein, particularly examples 16-19, wherein the plurality of distally extending protrusions extend at an oblique angle relative to a longitudinal axis of the anchoring dilator.

Example 21: A surgical dilator system according to any example herein, particularly examples 16-20, wherein the plurality of distally extending protrusions have lengths of 4 mm.

Example 22: A surgical dilator system according to any example herein, particularly examples 16-21, wherein the proximal portion of the anchoring dilator comprises a handle.

Example 23: A surgical dilator system according to any example herein, particularly examples 16-22, further comprising an introducer extending between a proximal portion and a distal portion and defining a central lumen extending longitudinally therethrough, wherein the central lumen of the introducer is sized and configured to receive the anchoring dilator.

Example 24: A surgical dilator system according to any example herein, particularly example 23, wherein the proximal portion of the introducer comprises a handle.

Example 25: A surgical dilator system according to any example herein, particularly examples 16-24, further comprising a universal dilator, the universal dilator extending between a proximal portion and a distal portion and defining a central lumen extending longitudinally therethrough, wherein the distal portion of the universal dilator comprises a tapered region that tapers toward a distal tip of the distal portion, and wherein the distal tip defines an opening in fluid communication with the central lumen.

Example 26: A surgical dilator system according to any example herein, particularly example 25, wherein the tapered region of the universal dilator defines a conical shape.

Example 27: A surgical dilator system according to any example herein, particularly examples 25-26, wherein the central lumen of the universal dilator is sized and configured to receive the guidance rod.

Example 28: A surgical dilator system according to any example herein, particularly examples 25-27, wherein the universal dilator further comprises a handle disposed at the proximal portion of the universal dilator.

Example 29: A kit for forming a surgical corridor in a patient, the kit comprising: a first tubular dilator extending between a proximal portion and a distal portion and defining a central lumen extending longitudinally therethrough, wherein the central lumen is sized and configured to receive a guidance rod; an anchoring dilator extending between a proximal portion and a distal portion and defining a central lumen extending longitudinally therethrough, wherein the distal portion comprises a plurality of distally extending protrusions for frictionally engaging patient tissue, wherein the anchoring dilator is disposed radially outward of the first tubular dilator; and a reamer comprising a longitudinal body extending between a proximal portion and a distal portion and defining a central lumen extending longitudinally therethrough, wherein the distal portion comprises a tapered region that extends between a base and a distal tip and tapers toward the distal tip, wherein at least a portion of the tapered region comprises a cutting edge for removing bone along the surgical corridor, and wherein the distal tip defines an opening in fluid communication with the central lumen, the central lumen configured to accommodate the guidance rod.

Example 30: A kit for forming a surgical corridor in a patient according to any example herein, particularly example 29, further comprising an introducer extending between a proximal portion and a distal portion and defining a central lumen extending longitudinally therethrough, wherein the introducer is disposed radially outward of the anchoring dilator.

Example 31: A method of forming a surgical corridor in a patient, the method comprising: inserting a guidance rod through an incision site and advancing the guidance rod toward a treatment location of a patient such that a distal tip of the guidance rod contacts tissue superficial relative to the treatment location, wherein the path of the guidance rod defines a surgical corridor; inserting a reamer over the guidance rod to form the surgical corridor, the reamer comprising a tapered region that tapers from a base of the tapered region toward a distal tip of the tapered region, the tapered region comprising a cutting edge; removing the reamer from the surgical corridor; advancing a first tubular dilator over the guidance rod, thereby expanding the surgical corridor and incision site; advancing an anchoring dilator over the first tubular dilator; and frictionally engaging the patient's tissue with a plurality of distally extending protrusions defined on a distal portion of the anchoring dilator.

Example 32: A method of forming a surgical corridor in a patient according to any example herein, particularly example 31, further comprising rotating the reamer such that the cutting edge removes tissue along the surgical corridor.

Example 33: A method of forming a surgical corridor in a patient according to any example herein, particularly examples 31-32, further comprising advancing one or more additional tubular dilators over the first tubular dilator, thereby further expanding the surgical corridor and incision site.

Example 34: A method of forming a surgical corridor in a patient according to any example herein, particularly examples 31-33, further comprising forming the surgical corridor comprises forming a surgical corridor through a facet joint of a patient.

Example 35: A method of forming a surgical corridor in a patient according to any example herein, particularly example 34, further comprising removing disc material from a disc space of the patient via one or more instruments advanced through the surgical corridor.

Example 36: A method of forming a surgical corridor in a patient according to any example herein, particularly example 35, wherein the surgical corridor intersects a middle aspect of the facet joint between an inferior articular process of a vertebra above the disc space and a superior articular process of a vertebra below the disc space.

Example 37: A method of forming a surgical corridor in a patient according to any example herein, particularly examples 34-35, wherein aspects of the inferior and superior articular processes are preserved on both sides of the surgical corridor so as to define lateral sides of the surgical corridor.

Example 38: A method of forming a surgical corridor in a patient according to any example herein, particularly examples 35-37, further comprising inserting an intervertebral disc into the disc space.

Example 39: A method of forming a surgical corridor in a patient according to any example herein, particularly examples 35-38, further comprising inserting a graft material into the disc space.

Example 40: A method of forming a surgical corridor in a patient according to any example herein, particularly examples 31-39, further comprising inserting a graft material into the surgical corridor.

Example 41: A method of forming a surgical corridor in a patient according to any example herein, particularly examples 35-40, wherein graft material is deposited into the surgical corridor from the disc space to the incision site.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

What is claimed:

1. A surgical reamer system comprising:
a reamer for forming a surgical corridor in a patient, the reamer comprising a longitudinal body extending between a proximal portion and a distal portion and defining a central lumen extending longitudinally therethrough,
wherein the distal portion comprises a tapered region that extends between a base and a distal tip and tapers toward the distal tip,
wherein at least a portion of the tapered region comprises a cutting edge for removing bone along the surgical corridor,
wherein the distal tip defines an opening in fluid communication with the central lumen.

2. The surgical reamer system of claim 1, wherein the cutting edge of the reamer extends helically about the tapered region.

3. The surgical reamer system of claim 1, wherein the cutting edge of the reamer extends along an entire length of the tapered region.

4. The surgical reamer system of claim 1, wherein the cutting edge of the reamer is axially spaced from the distal tip.

5. The surgical reamer system of claim 1, wherein the distal portion of the reamer defines a groove extending longitudinally along an outer surface of the distal portion,
wherein the groove extends through at least a portion of the cutting edge.

6. The surgical reamer system of claim 5, wherein the groove extends to the base of the tapered region.

7. The surgical reamer system of claim 1, wherein the longitudinal body of the reamer comprises a barreled portion having a diameter greater than a diameter of the base of the tapered region.

8. The surgical reamer system of claim 1, wherein the reamer further comprises a handle for rotating the reamer about a longitudinal axis.

9. The surgical reamer system of claim 1, further comprising:
a joint stripper for removing soft tissue from a patient's joint, the joint stripper comprising a longitudinal body extending between a proximal portion and a distal portion and defining a central lumen extending longitudinally therethrough,
wherein the distal portion of the joint stripper comprises a plurality of distally extending protrusions, each protrusion comprising a cutting edge for removing soft tissue from the patient's joint.

10. The surgical reamer system of claim 9, wherein the joint stripper further comprises a handle for rotating the joint stripper about a longitudinal axis.

11. The surgical reamer system of claim 9, wherein the joint stripper is disposable within an anchoring dilator, the anchoring dilator comprising:
a proximal portion and a distal portion and defining a central lumen extending longitudinally therethrough, wherein the distal portion comprises a plurality of distally extending protrusions for frictionally engaging patient tissue.

12. A surgical dilator system for enlarging a corridor in a patient, the surgical dilator system comprising:
a guidance rod extending between a proximal end and a tapered distal tip;
a first tubular dilator extending between a proximal portion and a distal portion and defining a central lumen extending longitudinally therethrough, wherein the central lumen is sized and configured to receive the guidance rod; and
an anchoring dilator extending between a proximal portion and a distal portion and defining a central lumen extending longitudinally therethrough, wherein the distal portion comprises a plurality of distally extending protrusions for frictionally engaging patient tissue, wherein the central lumen of the anchoring dilator is sized and configured to receive the guidance rod and first tubular dilator.

13. The surgical dilator system of claim 12, wherein the surgical system further comprises one or more additional tubular dilators disposed radially outward of the first tubular dilator.

14. The surgical dilator system of claim 12, wherein the plurality of distally extending protrusions comprise a plurality of serrated edges.

15. The surgical dilator system of claim 12, wherein the plurality of distally extending protrusions extend circumferentially about the distal portion of the anchoring dilator.

16. The surgical dilator system of claim 12, wherein the plurality of distally extending protrusions extend at an oblique angle relative to a longitudinal axis of the anchoring dilator.

17. The surgical dilator system of claim 12, further comprising an introducer extending between a proximal portion and a distal portion and defining a central lumen extending longitudinally therethrough, wherein the central lumen of the introducer is sized and configured to receive the anchoring dilator.

18. The surgical dilator system of claim 17, wherein the proximal portion of the introducer comprises a removable handle.

19. The surgical dilator system of claim 12, further comprising a universal dilator, the universal dilator extending between a proximal portion and a distal portion and defining a central lumen extending longitudinally therethrough,
wherein the distal portion of the universal dilator comprises a tapered region that tapers toward a distal tip of the distal portion, and
wherein the distal tip defines an opening in fluid communication with the central lumen.

20. The surgical dilator system of claim 19, wherein the central lumen of the universal dilator is sized and configured to receive the guidance rod.

21. The surgical dilator system of claim 19, wherein the universal dilator further comprises a handle disposed at the proximal portion of the universal dilator.

22. A kit for forming a surgical corridor in a patient, the kit comprising:
a first tubular dilator extending between a proximal portion and a distal portion and defining a central lumen extending longitudinally therethrough, wherein the central lumen is sized and configured to receive a guidance rod;
an anchoring dilator extending between a proximal portion and a distal portion and defining a central lumen extending longitudinally therethrough, wherein the distal portion comprises a plurality of distally extending protrusions for frictionally engaging patient tissue, wherein the anchoring dilator is disposed radially outward of the first tubular dilator; and
a reamer comprising a longitudinal body extending between a proximal portion and a distal portion and defining a central lumen extending longitudinally therethrough,
wherein the distal portion comprises a tapered region that extends between a base and a distal tip and tapers toward the distal tip,
wherein at least a portion of the tapered region comprises a cutting edge for removing bone along the surgical corridor, and
wherein the distal tip defines an opening in fluid communication with the central lumen, the central lumen configured to accommodate the guidance rod.

23. The kit of claim 22, further comprising an introducer extending between a proximal portion and a distal portion and defining a central lumen extending longitudinally therethrough, wherein the introducer is disposed radially outward of the anchoring dilator.

24. A method of forming a surgical corridor in a patient, the method comprising:
inserting a guidance rod through an incision site and advancing the guidance rod toward a treatment location of a patient such that a distal tip of the guidance rod contacts tissue superficial relative to the treatment location, wherein the path of the guidance rod defines a surgical corridor;
inserting a reamer over the guidance rod to form the surgical corridor, the reamer comprising a tapered region that tapers from a base of the tapered region toward a distal tip of the tapered region, the tapered region comprising a cutting edge;
removing the reamer from the surgical corridor;
advancing a first tubular dilator over the guidance rod, thereby expanding the surgical corridor and incision site;
advancing an anchoring dilator over the first tubular dilator; and
frictionally engaging the patient's tissue with a plurality of distally extending protrusions defined on a distal portion of the anchoring dilator.

25. The method of claim 24, further comprising forming the surgical corridor comprises forming a surgical corridor through a facet joint of a patient,
wherein the surgical corridor intersects a middle aspect of the facet joint between an inferior articular process of a vertebra above the disc space and a superior articular process of a vertebra below the disc space,
wherein aspects of the inferior and superior articular processes are preserved on both sides of the surgical corridor so as to define lateral sides of the surgical corridor.
